(19) United States
(12) Patent Application Publication
(10) Pub. No.: US 2007/0042433 A1
(43) Pub. Date: Feb. 22, 2007

(54) Neuregulin protein regulation of synaptic proteins

(76) Inventor: Jianxin Bao, St. Louis, MO (US)

Correspondence Address: POLSINELLI SHALTON WELTE SUELTHAUS P.C., 700 W. 47TH STREET, SUITE 1000, KANSAS CITY, MO, 64112-1802, US

(21) Appl. No.: 10/550,673

(22) PCT Filed: Mar. 26, 2004
(86) PCT No.: PCT/US04/09499

§ 371 Date: Jun. 14, 2006

(30) Foreign Application Priority Data
Mar. 26, 2003 (US) 60457321

(51) Int. Cl. G01N 33/567; C07H 21/04; C12P 21/04; C07K 14/705

(52) U.S. Cl. 435/7.2; 435/69.7; 435/320.1; 435/325; 530/350; 536/23.5

(57) ABSTRACT

Methods are provided for discovery of new lead pharmaceuticals useful for addressing diseases and other problems of hearing. Compounds that modulate PSD-95 induction are discovered via use of the Nrg-1/Eos signaling pathway. Cell based assay systems are particularly described that identify modulation of binding of Nrg-ICD with an Eos binding site. Other features include the promotion and/or inhibition of Nrg-ICD translocation across nuclear membranes.

Human Nrg-ICD Polypeptide Sequence

SEQ ID NO: 1

KTKKQRKKLHDRLRQSLRSERNNVMNMANGPHHPNPPPDNVQLVNQYVSK
NIISSERVVERETETSFSTSHYTSTTHHSMTVTQTPSHSWSNGHTESILS
ESHSVLVSSSVENSRHTSPTGPRGRLNGIGGPREGNSFLRHARETPDSYR
DSPHSERYVSAMTTPARMSPVDFHTPTSPKSPPSEMSPPVSSLTISIPSV
AVSPFMDEERPLLLVTPPRLREKYDNHLQQFNSFHNNPTHESNSLPPSPL
RIVEDEEYETTQEYEPAQEPPKKLTNSRRVKRTKPNGHISSRVEVDSDTS
SQSTSSESETEDERTGEDTPFLSIQNPMATSLEPAAAYRLAENRTNPANR
FSTPEELQARLSSVIANQDPIAV

This application claims priority to U.S. provisional application No. 60/457,321 filed Mar. 26, 2003, the entirety of which is hereby incorporated by reference.

### FIELD OF THE INVENTION

The invention relates generally to control systems and pharmaceuticals for controlling synapse functioning and more specifically to methods, and materials for finding and using pharmaceuticals that alter PSD-95 and other components of synapse functioning.

### BACKGROUND

The transmembrane protein Neuregulin-1 (“Nrg-1”) originally was identified as a 44-kD glycoprotein that interacts with the NEU/ERBB2 receptor tyrosine kinase to increase phosphoiylation of the glycoprotein's tyrosine residues. At least 15 isoforms can be produced from the Nrg1 gene by alternative splicing. These isoforms are tissue-specifically expressed and differ significantly in their structures. The major expressed form in the nervous system contains a highly conserved intracellular domain that has three splicing isofroms that are named the A, B, and C forms. The longest A form likely is the major isoform expressed in the nervous system. In the extracelluar domains of all HRG isoforms contain an epidermal growth factor-like (EGF-like) sequence. The receptors for Nrg-1 isoforms are the ERBB family of tyrosine kinase transmembrane receptors. Through interaction with ERBB receptors, Nrg1 isoforms induce the growth and differentiation of epithelial, neuronal, glial, and other types of cells.

Recent research results suggest that the cytoplasmic portion of Nrg has a role in apoptosis, as reported by Grimm et al. in “Neu Differentiation Factor (NDF), a Dominant Oncogene, Causes Apoptosis In Vitro and In Vivo” J. Exp. Med., 188: No. 8 1535-1539 (1998). Furthermore, a basic 157 amino acid sequence common to the cytoplasmic tails of all transmembrane neuregulin isoforms has been identified that interacts with LIMK1. Presumably this interaction causes phosphorylation of the cytoplasmic portion of Nrg. The role of a 217 amino acid portion remains unknown, however.

Much research has focused on the Nrg protein because the protein may be involved in synaptic formation and maintenance, which is basic to proper brain functioning. However, no clear idea has emerged for how this protein is involved in synapse functioning. Any new information regarding this system should result in new treatment modalities for nerve function related diseases, as well as chronic diseases of aging.

### SUMMARY OF THE INVENTION

One embodiment of the invention is a drug discovery method for identifying a compound (or compounds) that modulates the induction of PSD-95 by the Nrg-1/Eos signaling pathway comprising contacting one or more test compounds with Nrg-ICD or a portion thereof, wherein the Nrg-ICD or portion thereof is encoded by a nucleic acid that hybridizes to a nucleic acid having SEQ ID NO: 1 in 5.times.SSC at 42.degree. C., and identifying the binding between the one or more test compounds and Nrg-ICD or a portion thereof.

Another embodiment is a drug discovery method for identifying a compound that modulates binding of Nrg-ICD with a binding site of Eos, comprising:
(i) contacting one or more test compounds with Nrg-ICD or a portion thereof and with at least one binding site of Eos, wherein the Nrg-ICD is encoded by a nucleic acid that hybridizes to a nucleic acid having SEQ ID NO: 1 in 5.times.SSC at 42.degree. C.;
(ii) contacting Nrg-ICD or a portion thereof with the at least one binding site of Eos in the absence of the one or more test compounds; and
(iii) identifying a difference in binding between Nrg-ICD or a portion thereof and with the at least one binding site of Eos between the contacting of (i) and the contacting of (ii).

Yet another embodiment is a drug discovery method for identifying a compound that modulates translocation of Nrg-ICD into a cell nucleus, comprising contacting a cell with one or more test compounds; and detecting movement of Nrg-ICD from the cell cytoplasm into the cell nucleus. Yet another embodiment is A method for identifying a compound that promotes or inhibits translocation of Nrg-ICD across the nuclear membrane of a cell, comprising: transgenically expressing in cells, a polypeptide complex comprising a nuclear localization sequence of Nrg-ICD and a regulatory marker, wherein the localization sequence of Nrg-ICD is at least 90% homogeneous with a portion that exceeds 20 amino acids of SEQ ID NO: 2 and the regulatory marker influences the expression of a gene when present within the nucleus of the cell; and contacting the cells with test compounds and determining whether a test compound affects translocation of Nrg-ICD across the nuclear membrane of the cell.

Still another embodiment is a method for identifying a compound that modulates the proteolysis of Nrg-1 to form Nrg-ICD, comprising incubating a cellular membrane form of Nrg-1 in the presence of the compound, and detecting the formation of a carboxylic end portion of Nrg-1 that is less than 60 kilodaltons in size. Yet another embodiment is a method for identifying a compound that modulates gene activity by binding to an Ikaous ½ sequence, comprising providing transgenic cells that contain a reporter gene operably coupled to a promoter that comprises Ikaous ½ sequence; contacting the cells with one or more test substances; and detecting the induction of the reporter gene in response to one or more test substances.

Yet another embodiment is a fusion polypeptide of a pharmaceutically active compound discovered by a method as described herein, comprising a first polypeptide portion of between 8 and 50 amino acids long that exhibits binding to Nrg-ICD or Eos and a second polypeptide portion comprising a transporter moiety of between 10 and 20 amino acids long. Yet another embodiment is a method for enhancing learning in an animal, comprising providing to the animal a compound that modulates the formation or translocation of Nrg-ICD into the nucleus of a nerve cell, wherein the compound is a fusion compound as described herein. Yet another is a method for preventing neuronal excitotoxicity in an animal, comprising providing to the animal a pharmaceutical that attenuates the nuclear signaling pathway of Nrg-1. Yet another embodiment is a transgenic animal with enhanced learning capability, produced by the process of stably incorporating an exogenous Neu-1 gene into the animal and expressing the gene. Yet another embodiment is an isolated protein complex, comprising primarily of Ng-ICD and Eos. Yet another embodiment is a vector that comprises a gene encoding Nrg-ICD and a gene encoding Eos. In an embodiment, the Nrg-1 has a sequence selected from known Nrg-1 sequences, including for example, the sequences comprising approximately 15 distinct structurally-related isoforms (Lemke, Mol. Cell. Neurosci. 7:247-262, 1996 and Peles and Yarden, BioEssays 15:815-824, 1993), isoforms of NRG-1 such as Neu Differentiation Factor (NDF; Peles et al., Cell 69, 205-216, 1992 and Wen et al., Cell 69, 559-572, 1992), Heregulin (HRG; Holmes et al., Science 256:1205-1210, 1992), Acetylcholine Receptor Inducing Activity (ARIA; Falls et al., Cell 72:801-815, 1993), and the glial growth factors GGF1, GGF2, and GGF3 (Marchionni et al. Nature 362:312-8, 1993). The contents of each cited reference, and particularly the genetic and amino acid sequences are specifically incorporated by reference.

Other embodiments readily will be appreciated by a skilled artisan upon reading the disclosure and the details in the references cited herein.

### FIGURES

FIG. 1 shows the amino acid sequence (SEQ ID NO: 1) of a human Nrg-1-ICD protein.

FIG. 2 shows a nucleic acid sequence (SEQ ID NO: 2) that encodes the amino acid sequence of FIG. 1 and that includes an open reading frame for this sequence.

FIG. 3 shows two representative nuclear localization sequences, (SEQ ID NO: 3, and SEQ ID NO:4).

FIG. 4 shows two alternative binding site sequences in Nrg-ICD for Eos, (SEQ ID NO: 5 and SEQ ID NO: 6).

FIG. 5 shows a human Eos DNA binding domain (SEQ ID NO: 7).

FIG. 6 shows a human Eos domain for binding to Nrg-ICD (SEQ ID NO: 8).

FIG. 7 shows three peptides that block Nrg-ICD/Eos signaling pathways in vivo (SEQ ID NO: 9, SEQ ID NO: 10, and SEQ ID NO: 11.)

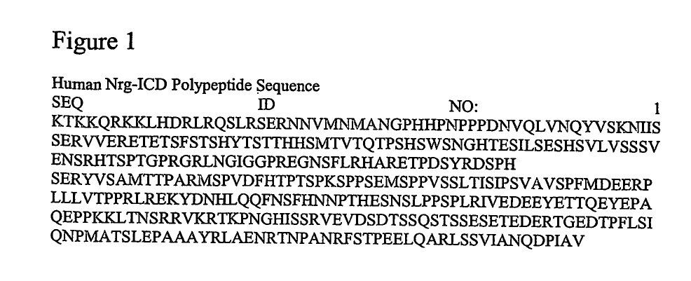

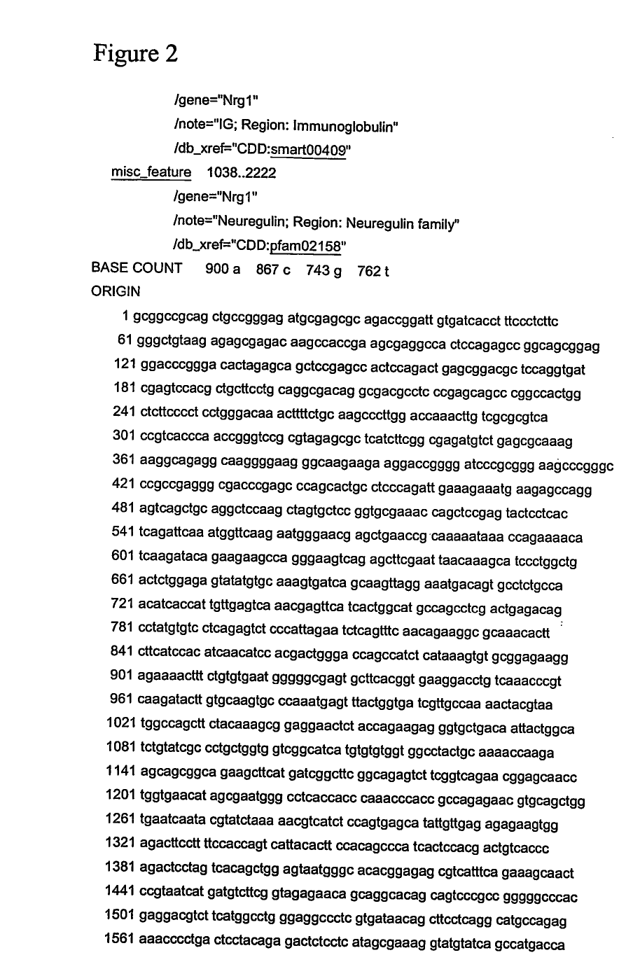

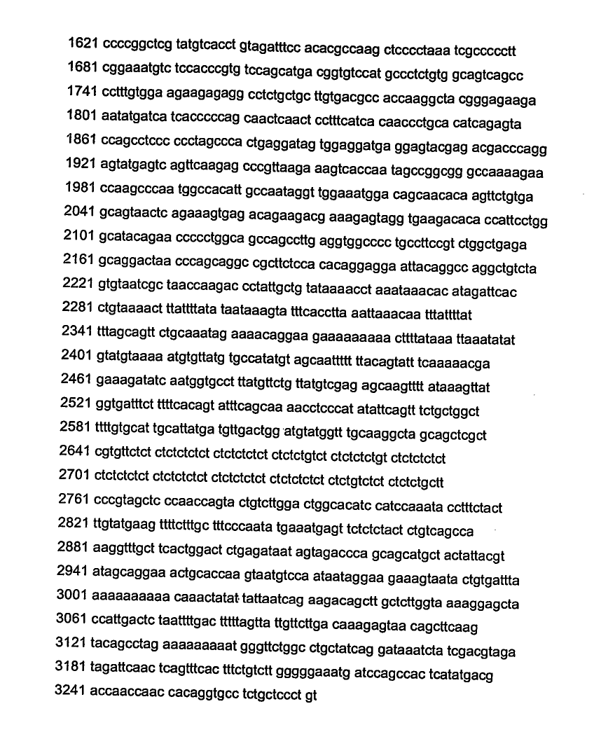

### DETAILED DESCRIPTION

The inventors discovered that the cytoplasmic portion of Nrg-1 enters the cell nucleus and regulates PSD-95 and other synaptic scaffolding protein transcriptions. Furthermore, the nuclear translocation of the Nrg-1 intracellular domain was found to be increased by neuronal activity. Moreover, this increase was shown to be accompanied by enhancement of PSD-95 protein through binding between the intracellular domain of Nrg-ICD and a zinc-finger protein of the transcription factor Eos, which was found to interact with the PDS-95 promoter and induce endogenous PSD-95 expression in both non-neuronal and neuronal cells. These discoveries were translated into several technologies of significant importance to biomolecular and cellular mechanisms of learning, memory, and other neurological processes.

A Variety of New Technologies

From these surprising findings, new drug development technologies are invented for discovering pharmaceutically active compounds that can improve neuronal adaptation and/or alleviate neuronal diseases. Such compounds may be used for improved learning, improved memory, and alleviation of diseases associated with neuronal malfunctioning. Additional discoveries based on these findings include the ability to engineer human and other animals with improved memory and learning capacity.

The discoveries can be used in various ways to create new materials and new methods that impact neuronal functioning. For example, chemical and biomolecular intervention may, for example, occur at the level of Nrg-ICD processing at the cell membrane, transit of Nrg-ICD into the nucleus, interaction of Nrg-ICD with other protein in the nucleus, synthesis of Eos and use of Eos. In each case, assays can be formulated for discovery of drugs that modulate the process. Genetic modifications to the neuron genome and other cell genomes may be carried out to effect changes at these points as well.

New sequences and localized portions of sequences have been discovered and found useful for many embodiments. In one group of embodiments, the gene promotion activity of Nrg-ICD is monitored, modified and/or otherwise exploited. The Nrg-ICD-Eos complex affects a specific promoter of the PSD-95 gene. That promoter may be coupled to another gene to allow regulation of another gene. Furthermore, Increased amount of Nrg-ICD and, (optionally), increased amount of Eos protein may be produced by up-regulating their genes and/or by transgenic expression leading to more of these protein(s).

The mechanisms of protein-based interactions discovered and explained herein also are a rich source for biochemical intervention. For example, the specific interaction between Nrg-ICD and a region of the zinc-finger transcription factor, Eos may be monitored, modified or otherwise exploited. This specific binding leads to increased expression of the PSD-95 gene. Modification to this binding with a specific compound or protein that may be supplied from outside the cell, or that may be supplied from expression within the cell, can modulate PSD-95 gene activity, with concomitant effects on memory and learning.

The translocation of Nrg-ICD into the nucleus can be monitored or even controlled in embodiments. Nrg-ICD translocation can be measured, for example, by monitoring changes in the amount in the cytoplasm and/or the nucleus. Nrg-ICD can be expressed as a conjugate with a marker such as green fluorescent protein. Nrg-ICD also can be detected more indirectly by, for example, immunolabelling. Yet another useful method is to couple the presence of Nrg-ICD in the nucleus with expression of a reporter gene. For example, a cell may be transformed with a reporter gene coupled to the PSD-95 promoter or other promoters containg one or more Ikarous sites. Translocation of Nrg-ICD to the nucleus may be monitored by expression of the reporter. In one embodiment a non-neuronal cell system, wherein the PSD-95 gene is silent, is used so that the reporter gene promoter does not compete with an active PSD-95 gene promoter. In another embodiment a transgenic cell is used that also expresses an Nrg-ICD and/or the Eos gene.

In sum, embodiments of the invention provide the ability to independently control the Nrg-ICD and Eos genes, as well as their protein products at a variety of control points. For example, the Eos gene may be altered or its protein functioning interfered with to affect PSD-95 transcription. Peptides that include portions of the Eos binding sites, preferably at least 8, 9, 10, 15, 20, 30, 40 or even longer portions, may be provided extracellularly, or intracellularly transgenically to compete with Eos for binding to Nrg-ICD or to DNA (see SEQ ID NO: 8 and SEQ ID NO: 7, respectively). Similarly, peptides that include portions of the Nrg-ICD binding sites, preferably at least 8, 9, 10, 15, 20, 30, 40 or even longer portions, may be provided extracellularly, or intracellularly transgenically to compete with Nrg-ICD for binding to Eos and/or DNA (see SEQ ID NO: 5, SEQ ID NO: 6). In a desirable embodiment a nucleic acid that is similar (at least 80%, 90%, 95% or higher homology) to either the Eos gene or the Nrg-ICD gene is provided to affect the respective gene. In some embodiments, two or more factors are used simultaneously for stronger control of PSD-95. Pharmaceutically active compounds may be discovered by assaying binding to portions of the Eos gene, the Eos protein, the Nrg-1 gene, the Nrg-1 protein, and also by assaying biological activity of the two proteins in response to presence of test compounds.

I. Drug Discovery

(i) Nrg-ICD Processing at the Cell Membrane

In an embodiment of the invention, phosphorylation of the cytoplasmic portion Neuregulin-1 growth factor is monitored to determine the effects of compounds on this process. Any technique that detects the incorporation of phosphate into the Neuregulin-1 growth factor may be used. In an embodiment, an assay that measures phosphate incorporation into this membrane protein within a cell is coupled with the exposure of the cell to one or more test compounds. For a control, preferably the same condition is used in the absence of a test compound.

In a desirable embodiment the incorporation of phosphate is further coupled with another event such as proteolysis of the protein or translocation of a carboxyl terminal fragment created by proteolysis into the nucleus. In one embodiment, proteolysis or phosphate incorporation is coupled with the incorporation of a reporter substance such as a fluorescent compound or atom and the reporter substance is used to monitor transit into the nucleus.

In another embodiment proteolytic processing of the cytoplasmic portion of the Neuregulin-1 growth factor is monitored to determine the effects of compounds on this process. Any technique that detects the formation of a smaller portion, such as a 53 kilodalton piece, or a 35 kilodalton piece may be used. For example, after incubation, cells may be lysed, water soluble (not membrane bound) molecules separated and the carboxyl terminal portion may be quantitated by immunoreaction with a suitable antibody conjugate. In a desirable embodiment the effect of a test compound on the proteolytic reaction may be tested by contacting the cells (or membrane fraction that contains the Neuregulin-1 growth factor) with test compounds. A control may be to contact cells with no compound, or with a compound that is not active in the system. Modulation of proteolysis may be determined by the effect of a compound on the amount of proteolytic product made.

Proteolytic processing converts the Nrg-1 protein to a smaller Nrg-1-CIP protein of about about 35 kilodaltons, in normal conditions. The term “about” in this context means with the regular precision accompanying gel electrophoresis determinations of protein size. For example, a protein of 25 kilo Daltons sometimes can be detected as 35 kilo Daltons. And a protein of 45 kilo Daltons sometimes can be detected as 35 kilo Daltons. In other instances such as a disorder that leads to non-natural processing, another size such as about 53 kilo daltons may be made.

(ii) Translocatioit of Nrg-ICD to the Nucleus

In an embodiment, the translocation of a carboxylic acid portion of the protein that is created by proteolysis from the cytoplasm to the nucleus is monitored. Such monitoring provides much information regarding neural processing and is very useful for drug discovery assays. A large variety of techniques may be used to monitor translocation. Some of these techniques are useful for large numbers of samples and are particularly desirable for drug discovery. For example, see U.S. Pat. No. 6,416,959 (System for cell-based screening), which describes systems, methods, screens, reagents and kits for optical system analysis of cells to rapidly determine the distribution, environment, or activity of fluorescently labeled reporter molecules for large scale screening. Also see U.S. No. 20020042366 (Method for treating inflammation), which describes a nuclear translocation assay that was used to identify transcription factor proteins that move from the cytoplasm to the nucleus by immunofluorescence. U.S. No. 20010041347 (System for cell-based screening) also teaches automated systems, methods, screens, and software that may be used to monitor movement of Nrg-ICD into the nucleus.

Yet another system that uses microbead-based test plates and test methods for fluorescence imaging systems is U.S. 20020098588, issued Jul. 25, 2002. Other relevant techniques are known to the skilled artisan, such as for example that taught by Feng Yang et al. in Analytical Biochemistry 266: 167-173 1999 (highly efficient green fluorescent protein based kinase substrates) and by Javier Farinas et al. in The Journal of Biological Chemistry 274: 7603-7606 1999 (Receptor mediated targeting of fluorescent probes in living cells). Each of these references, and particularly the materials and methods referred to, are incorporated by reference in their entireties as space and time limitations prevent importing further detail from these references.

Detecting translocation into the nucleus is particularly useful for drug discovery assays. Typically, a cell is contacted with solution that contains one or more test substances and the amount of Nrg-ICD in cytoplasm, nucleus or both is determined to find out a relationship between a test substance and the amount of Nrg-ICD that moves into the nucleus. A positive correlation with a test compound (increase in amount found in the nucleus) may indicate an effect of the compound on generation of Nrg-ICD, release of Nrg-ICD, stability of Nrg-ICD translocation of Nrg-ICD or the like. Such positive correlation may reveal that the compound can stimulate a neurological process such as memory or learning, generally through the up regulation of PSD-95.

(iii) Binding Between Nrg-ICD aid Eos to Form a Nrg-ICD-Eos Complex

In another embodiment binding between Nrg-ICD and human Eos is monitored. In one such embodiment the binding affinity is quantified and used to compare different Nrg-ICD's having different sequences. For example, a range of Nrg-ICD's from different animals and having different sequences are known. See, for example the sequence information provided in references cited herein. Despite the sequence differences, each Nrg-ICD that has at least some binding to human Eos has utility for drug test compound screening purposes. Preferably a sequence or sequence fragment is homologous enough to Nrg-ICD such that the protein or peptide binds to Eos with an affinity of at least 5% of the affinity of human Nrg-ICD of 35 kilo Dalton size. More preferably a sequence or sequence fragment shares enough homology such that the sequence or sequence fragment has at least 10% of the affinity of human Nrg-ICD to the Eos. Yet more preferably the homology is closer and provides at least 25% or even at least 50% or the affinity of human Nrg ICD. Thus, the xenopus Nrg-ICD sequence can be used for the discovery of drugs that are active in humans.

Binding measurements can be carried out by a large number of procedures. For example equilibrium dialysis may be carried out. Bound from non-bound fractions may be separated by column chromatography, and bound from non-bound phases may be separated by regular solid phase separation techniques. Identification of bound complex likewise may be achieved by use of a reportable label or labels on the components. In one high throughput multi-sample assay, fluorescence resonance energy transfer is used to detect when a first fluorescent probe on Nrg ICD or fragment thereof, is close to a second probe on Eos.

In an embodiment the binding between Nrg-ICD and Eos is probed in the presence of one or more test compounds as a drug screening assay. Generally, the two molecules are incubated in the presence of one more test compounds to determine whether the test compound facilitates or inhibits binding. A compound that affects binding may be selected as a lead compound for affecting a neurological system such as memory, learning, hyperstimulation and the like.

In another embodiment, an assortment of genes are prepared that encode modifications of the Nrg-ICD sequence or fragment of the Nrg-ICD sequence. A combinatorial library selection procedure is used to select one or more nucleic acid sequences that bind with greater binding affinity to Eos. Such techniques are known, as exemplified in U.S. Pat. Nos. 5,807,683 (Combinatorial libraries and methods for their use); U.S. Pat. No. 5,738,996 (Combinational library composition and method); U.S. Pat. No. 6,518,065 (Methods for generating polynucleotides having desired characteristics by iterative selection and recombination); U.S. Pat. No. 6,172,197 (Methods for producing members of specific binding pairs); U.S. Pat. No. 6,132,970 (Methods of shuffling polynucleotides); and U.S. Pat. No. 6,007,988 (Binding proteins for recognition of DNA), the contents of which are specifically incorporated by reference in their entireties. Using one of these or other known procedures, an improved Nrg-ICD sequence may be designed having greater affinity for Eos. Alternatively, an improved Eos may be designed having greater affinity for Nrg-ICD. In another embodiment one or more improved proteins is further tested to determine its effect on transcription of the PSD-95 protein.

(iv) Binding Between Nrg-ICD-Eos Complex and a Gene Promoter

It was discovered that an Nrg-ICD-Bos complex up regulates the PSD-95 gene through interaction with the promoter of this gene. In an embodiment this binding is assayed and used to detect a compound having pharmaceutical activity. One or more test compounds are incubated in the presence of a cell or cell free system that comprises the Nrg-ICD-Eos complex and the PSD-95 gene promoter. Binding between the complex and the promoter is detected and used to determine whether one or more test compounds may have pharmaceutical activity. In a particularly desirable embodiment the PSD-95 promoter is operably linked, and preferably cis-linked to a reporter gene that creates a detectable signal after the gene's transcription.

(v) Cell Culture Screening of Compounds

Compounds may be screened by looking at their effects on one or more aspects of the PSD-95 control system in cells. For example, PSD-95 synthesis, or capability to regulate PSD-95 synthesis, may be monitored in transgenic cells that utilize a reporter gene coupled to one or more Ikarous sites. Preferably, the one or more Ikarous sites comprises a specific Ikarous sequence of the PSD-95 promoter. Upon increased Nrg-ICD, increased translocation, or increased binding of Nrg-ICD-Eos to DNA etc., the transcription of a reporter gene that has this promoter will be increased, causing increased amount of the reporter gene product. The increased amount may be determined optically or by any other relevant method.

Large scale cell detection systems may be used in a wide variety of configurations. Typically, individual cells, or groups of cells are cultured and exposed to one or more test substances. An index of cellular health or some other biochemical or biological response is monitored and correlated with the presence of specific compounds. For example, cell death may be detected fluoroscopically, as for example taught in U.S. Pat. No. 5,534,416 (Fluorescent viability assay using cyclic-substituted unsymmetrical cyanine dyes) and other references cited therein. A variety of cell manipulation and assay technologies are available, such as those promoted by Axiom Technologies, Inc. of San Diego (see U.S. Pat. No. 5,804,436); Caliper Technologies Corporation of Palo Alto (see U.S. Pat. Nos. 5,942,443; 6,001,231; 6,046,056; 6,132,685; 6,149,787; and 6,529,835). A variety of cell imaging systems are known and particularly useful, as exemplified by U.S. Pat. No. 5,627,643 (Method and detector for separation processes) and the citations therein. Each of these cited patents specifically is incorporated by reference in its entirety, and more specifically, the materials and methods for manipulating cells and detecting changes in the cells, for application to embodiments of the invention. Due to space and time limitations, further details from these 9 patents and further patents cited therein are not described further.

(vi) Screening Compounds In Vivo

As an initial screen, and also as a later screen following mass screening of large numbers of compounds, an in vivo test assay is particularly desirable. In one such embodiment a test chemical is linked with a short peptide that facilitates cell entry. The conjugate is introduced into an animal body by, for example, injection intraperitoneally, into the brain or other lumen. After waiting a period of time for cell entry and effect a response is measured. The waiting time typically may be from about 1 minute to 10 days, preferably from 15 minutes to 2 days, and more preferably from about 30 minutes to one 5 day. The effect of the conjugate may be determined on the peripheral nervous system such as dorsal ganglion or the central nervous system. Any index of biological, biochemical, physiological, or behavioral response may be determined. For example, PSD-95 induction in dorsal ganglion may be determined after pain stimulation; PSD-95 induction in spiral ganglia may be determined after noise stimulation; and PSD-95 induction in the hippocampus may be determined after learning. Other assays may be used, such as measuring neuronal cell death after environmental stimulation, to show protection against overstimulation.

In a favorable embodiment, a peptide having a sequence SEQ ID NO: 9; SEQ ID NO: 10; SEQ ID NO: 11, or a derivative thereof is used in vivo for altering PSD-95. These peptides comprise a cellular HIV-1 permeater moiety. A variety of sequences may be used for modifying PSD-95 through cell entry in this fashion. Other sequences as described herein may be used. Conservative amino acid substitutions corresponding up to 1%, 2%, 5%, 10%, 15%, 20% or even more of a peptide sequence may be used without completely abolishing activity and are desirable.

II. Therapy/Prophylaxis

(i) Increase Neuregulin-1 and/or Eos or Improved Memory/Learning

In an embodiment of the invention, the amount of precursor to Nrg-ICD in a cell is increased. This increase leads, directly or indirectly to improved memory and/or learning performance of an animal that contains nerve cells with the increased precursor. Without wishing to be bound by any one theory for this embodiment of the invention it is believed that at least in some instances, memory and/or learning is limited by the amount of PSD-95 in synapse generating cells. The amount of PSD-95 can be increased by upregulating PSD transcription. The increased PSD-95 provides faster and/or greater strengthening of synaptic transmission in response to signals that transit the synapse junction. Because of the greater response, less stimuli is needed to effect a change in the synapse. The lesser stimuli needed means that less effort is required to remember or to learn something.

In an analogous way, the amount of Eos available can be modified as well. The increase in Eos can lead to improved memory, and/or learning performance. In some individuals, a limitation in the amount or quality of Eos is rectified by introducing more of this protein into cells, either endogenously (by protein synthesis within the cell) or (less preferably) exogenously.

A skilled molecular biologist can increase Neuregulin-1 and/or Eos several ways that will be apparent to a reader. For example, foreign Neuregulin and/or Eos gene(s) may be added transgenically. Homologous recombination also may be carried out by incorporating a constitutive or other strong promoter or enhancer to upregulate a native (existing in the cell) Neuregulin or Eos gene. A variety of genetic transformation techniques and vectors may be used for this purpose, as described below.

In another embodiment, one or more extracellular effectors cause proteolysis of Nrg-1 to form Nrg-ICD. A drug screening assay may be carried out wherein detection of ICD formation (transport into the nucleus, upregulation of PSD-95 or other gene such as a reporter gene) is monitored in cells. Cells are contacted with different test compounds and the effects are correlated to determine whether a compound affects activity.

In a desirable embodiment one or more extra Eos genes are added (or up regulated) and one or more Neuregulin-1 genes are added (or up regulated) to further facilitate an increase in Nrg-ICD-Eos complex for up regulating the PSD-95 gene. In yet another embodiment one or more PSD-95 genes also are added to help increase the amount of PSD-95 expression. Accordingly, embodiments of the invention include vectors that comprise one or more such genetic elements and also cells and transgenic animals that contain one more such genetic elements. A desirable embodiment provides a vector that comprises both a PSD-95 gene and a Neuregulin-1 gene.

A particularly desirable embodiment is a method for improving nerve cell function comprising adding one or more genes as detailed herein via a vector to cells that contain excitatory membrane or that develop into cells that contain excitatory membranes. The gene(s) preferably are added via a vector such as a viral vector. The cells may be cultured cells and later added to an animal. Alternatively, the cells may exist in vivo and the method provides improved functioning of a pre-existing nervous system.

(ii) Decrease Neuregulin-1 for Treatment of Ischemic Brain Damag, Overstimulation

In some embodiments, materials and methods are used to down-regulate PSD-95. As described, for example, by Aarts et al. in Science, Vol. 298 pp. 846-850 (2002) down regulation or targeting of PSD-95 protein represents an alternative therapy for diseases that involve excitotoxicity and ischemic brain damage. Embodiments of the invention can inhibit the synthesis of PSD-95. Interference of the synthesis or functioning of Nrg-1 or of Nrg-ICD may be used to down regulate PSD-95 for desirable clinical effects.

Most desirably, materials and methods may be used to prevent neuronal death after damage, such as noise in the spiral ganglion, injury in the spinal cord and brain, or chronic pain from a dorsal root ganglion or spinal cord. In an embodiment, a substance that inhibits the formation if Nrg-ICD; translocation of Nrg-ICD into the nucleus; binding of Nrg-ICD to Eos; formation of Eos; and/or binding of Nrg-ICD-Eos to the PSD-95 promoter is used prior to, immediately after or at longer times after an event leading to neuronal stress. In an embodiment, the substance is administered to a person prophylactically at least 1 second, 1 minute, 5 minutes, 1 hour, 4 hours, a day, or even a longer period prior to an expected exposure to neuronal stress, such as high noise level, impact injury (such as boxing) to the brain, or other stress. In another embodiment the substance is administered long term (at least once daily, weekly or monthly) to counteract chronic pain. In an embodiment the substance is administered by injection and in another embodiment the substance is administered orally. The effect of administration is to desensitize neurons to overstimulation (or to remove oversensitivity to normal stimulation) at affected neurons, and thereby minimize cell death.

(iii) Peptides for Down-Regulating the PSD-95 Induction System

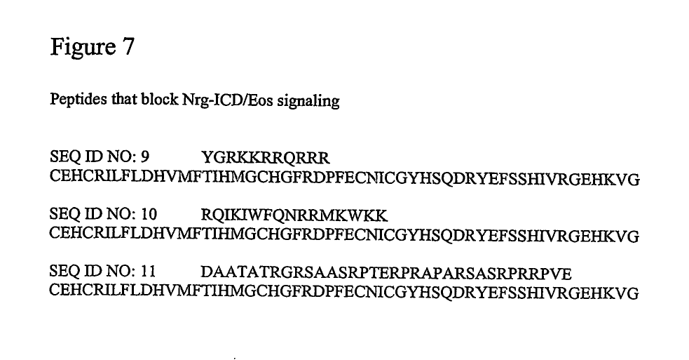

In some disease processes and physiological states an excess of stimulation and/or other perturbations in the overall biochemical system of PSD-95 induction by Nrg-ICD is alleviated by one or more peptide agents. In an embodiment, the peptide corresponds to a continuous sequence of at least 8 amino acids selected from any of SEQ ID NO: 1, 3, 4, 5, 6, 7, 8, 9, 10, and 11. In a desirable embodiment, the peptide is fused to a cellular entry factor such as the HIV-1 Tat residues 38 to 48 outside the transduction domain, as for example, described in Aarts et al. Science 298: 846-850 (2002) and Mann et al. EMBO J. 10: 1733 (1991). In a particularly desirable embodiment the peptide comprises a sequence SEQ ID NO: 9, SEQ ID NO: 10, SEQ ID NO: 11 (see FIG. 7) or a modification of such sequence.

(iv) Increase Translocation of Nrg-ICD for Enhanced PSD-95 Induction

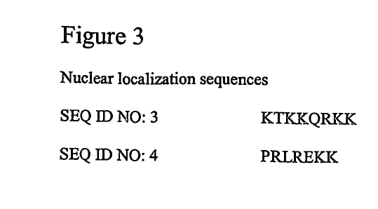

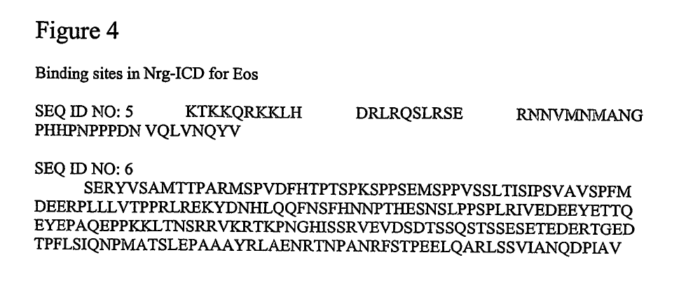

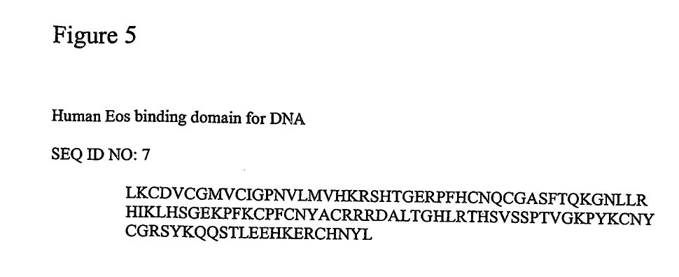

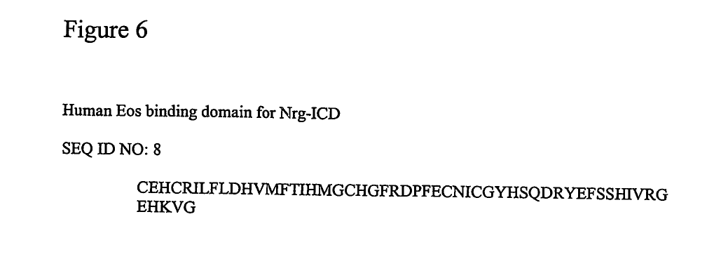

In another desirable embodiment, a modified Nrg-ICD is created that translocates into the nucleus more readily. In one embodiment, an extra segment of translocation sequence is added to the carboxyl terminal region, and desirably to the carboxy terminal end of the Neuregulin-1 gene. Examples of such translocation sequences are SEQ ID NO: 3 and SEQ ID NO: 4 provided in FIG. 3. A result of the increased translocation is increased PSD-95 synthesis, leading to improved synapse performance in many instances. In an embodiment, either sequence is added to another protein to direct translocation of that protein into the nucleus. In yet another embodiment, the two sequences are used in their relative position along the protein to be transcribed to direct a protein into the nucleus. In another embodiment two or more copies of a translocation sequence are added to a protein or to a gene encoding a protein.

(v) Increase Proteolysis of Neuregulin-1 for Increased Nrg-ICD Production

In another desirable embodiment, a modified Neuregulin-1 is produced that has at least one extra proteolytic site for enhanced sensitivity to proteolysis. In an embodiment, the site comprises one of the sequences: QRKK; QRKL: SPHS: and SPHE. In one embodiment one extra site containing one or more of these sequences is added. In another embodiment one, two, three, four, five or more amino acids such as glycine, serine, threonine or alanine are added between proteolysis sites to move a proteolysis spot away from the transmembrane region and from other sites.

In an embodiment two, three, four or even more proteolysis sites that comprise at least 6 continuous amino acids centered at the middle of regions that contain QRKK, QRKL, SPHS, or SPHE, are added adjacent to (on the carboxyl terminal side or on the amino terminal side of) the naturally occurring site. By “middle” is meant the point between the second and third amino acid of each group of four. In an embodiment the added site(s) are separated from adjacent sites by one, two, three, four, five, six, seven, eight, nine, ten or even more amino acid spacers. In another embodiment at least 6, 7, 8, 9 or even more than 10 continuous amino acids centered at the middle are added. Preferably a spacer comprises one or more small neutral amino acids such as glycine, alanine, serine, threonine, and the like.

(vi) Increase Eos Binding Sites for Improved Complex Formation

In another desirable embodiment, a modified Neuregulin-1 is produced that has more than one copy of the Eos binding site at its carboxyl terminus region. In an embodiment two, three, four or even more Eos binding sites that comprise at least 15, 20, 25, 30 or more continuous amino acids from SEQ ID NO: 5 and/or SEQ ID NO: 6 are added adjacent to (on the carboxyl terminal side or on the amino terminal side of) the naturally occurring site. In an embodiment the added site(s) are separated from adjacent sites by one, two, three, four, five, six, seven, eight, nine, ten or even more amino acid spacers. In another embodiment at least 35, 40, 50, 9 or even 60 continuous amino acids of the Eos binding site are added. Preferably a spacer comprises one or more small neutral amino acids such as glycine, alanine, serine, threonine, and the like.

### III. Materials for Embodiments of the Invention

A wide variety of cell types, animals, vectors, nucleic acids, and proteins may be used for embodiments of the invention. In particular, for drug discovery assays, a comparative test result most often is obtained to determine whether a test compound has activity. Accordingly, such tests do not require complete and exact human sequences of protein or nucleic acid. In an embodiment, an Nrg-ICD protein (or Eos protein) that binds at least 1%, 2%, 5%, 10%, 20%, or at least 50% (determined as an affinity constant) as well to its cognate binding site compared to an exact and complete human sequence may be used for drug discovery tests. As seen in cited references, for example, the cytoplasmic domain sequences obtained from different species typically have more than 70%, 80%, 85%, 87%, 90%, 95%, 97% or even greater homology. Each of these sequences displays measurable activity in one or more assays or tests described herein. Furthermore, other variations of sequences also lead to protein having desirable activity.

Cells

Many embodiments utilize intact cell synthesis of Neuregulin-1 and the modulation of PSD-95 protein. Many of the desirable cells are obtained from a kinder or brain cell lineage. In many instances, a protease in the cell acts upon neuregulin-1 to generate Nrg-ICD on the cytoplasmic side of the cell membrane. The Nrg-Icd enters the cell nucleus and upregulates PSD-95 or another gene. For assays and other methods that utilize one or more of these events, it is particularly preferred to use a cell that normally expresses Eos and preferably PSD-95 as well or another regulated protein having a promoter that responds to the Nrg-ICD-Eos complex. Preferred cell lines are 293T and 293S. Other cell lines that have endogenous Eos are particularly desirable because only Nrg-1 need to be expressed in certain embodiments that utilize these cells.

Transgenic Animals

In embodiments of the invention one or more genes are introduced into and stably expressed in an animal such as a human, mouse, canine, monkey, rat, ovine, bovine or other animal. In desirable embodiments a Neuregulin-1 gene such as the human gene is introduced into such animal and results in improved synaptic functioning. In another embodiment a normal human gene is introduced into a human cell that contains a gene that is genetically abnormal, and improves cellular function. In another embodiment, both Neuregulin-1 and PSD-95 genes are introduced. In yet another embodiment, both Neuregulin-1 and Eos genes are introduced. In yet another embodiment, a polypeptide sequence as described herein is expressed transgenically. A transgenic animal produced thereby can be used, for example as a test system for evaluating compounds for pharmaceutical effect.

Vectors

A variety of vectors may be use for transfer of genetic information into a cell according to embodiments of the invention. The term “vector” as used herein refers to a construction comprised of genetic material designed to direct transformation of a targeted cell. A vector contains multiple genetic material, preferably contiguous fragments of DNA or RNA, e.g., DNA derived from a plasmid, cosmid, phasmid or bacteriophage or synthesized by chemical or enzymatic means, positionally and sequentially oriented with other necessary elements such that the nucleic acid can be transcribed and when necessary translated in the transfected cells. The vector can contain one or more unique restriction sites for this purpose, and may be capable of autonomous replication in a defined host or organism such that the cloned sequence is reproduced. The vector may have a linear, circular, or supercoiled configuration and may be complexed with other vectors or other materials for certain purposes. The components of a vector can include but are not limited to a DNA molecule incorporating: (1) a sequence encoding a therapeutic or desired product; and (2) regulatory elements for transcription, translation, RNA stability and replication.

In embodiments, a preferred vector comprises the following elements linked sequentially at an appropriate distance to allow functional expression: a promoter, a 5′ mRNA leader sequence, a translation initiation site, a nucleic acid cassette containing the sequence to be expressed, a 3′ mRNA untranslated region, and a polyadenylation signal sequence. As used herein the term “expression vector” refers to a DNA vector that contains all of the information necessary to produce a recombinant protein in a heterologous cell.

In addition, the term “vector” as used herein can also include viral vectors, although non-viral vectors are preferred. A “viral vector” in this sense is one that is physically incorporated in a viral particle by the inclusion of a portion of a viral genome within the vector, e.g., a packaging signal, and is not merely DNA or a located gene taken from a portion of a viral nucleic acid. Thus, while a portion of a viral genome can be present in a vector of the present invention, that portion does not cause incorporation of the vector into a viral particle and thus is unable to produce an infective viral particle.

A vector as used herein can also include DNA sequence elements which enable extra-chromosomal (episbmal) replication of the DNA. Vectors capable of episomal replication are maintained as extra-chromosomal molecules and can replicate. These vectors are not eliminated by simple degradation but continue to be copied. These elements may be derived from a viral or mammalian genome. These provide prolonged or “persistent” expression as described below.

The vector can be used to provide expression of a nucleic acid sequence in tissue. In the present invention this expression preferably is enhanced by providing stability to an mRNA transcript from the nucleic acid sequence and/or secretion of the therapeutic protein. Expression includes the efficient transcription of an inserted gene or nucleic acid sequence within the vector. Expression products may be proteins including but not: limited to pure protein (polypeptide), glycoprotein, lipoprotein, phosphoprotein, or nucleoprotein. Expression products may also be RNA. The nucleic acid sequence is contained in a nucleic acid cassette. Expression of the nucleic acid can be continuous or controlled by endogenous or exogenous stimuli.

The term “persistent expression” as used herein refers to introduction of genes into the cell together with genetic elements which enable episomal (i.e., extrachromosomal) replication. This can lead to apparently stable transformation of the cell without the integration of the novel genetic material into the chromosome of the host cell.

“Stable expression” as used herein relates to the integration of genetic material into chromosomes of the targeted cell where it becomes a permanent component of the genetic material in that cell. Gene expression after stable integration can permanently alter the characteristics of the cell and its progeny arising by replication leading to stable transformation.

Proteins

The term “Neuregulin-1 refers to a protein having a sequence with homology to the human Neuregulin-1 protein sequence and that has at least some biological activity of the human Neuregulin-1 protein. Preferably the homology is at least 80%, more preferably at least 85%, 90%, 95% or even more than 97%. Homology is determined as percent amino acid identity when the sequence is lined up side by side with the human sequence. In an embodiment, Neuregulin-1 is a naturally occurring homologue (genetic variation found in nature) of Neuregulin-1 such as a variant of human Neuregulin-1 or of mouse Neuregulin-1.

The term “Nrg-ICD” refers to a carboxyl terminal sequence portion of Neuregulin-1 that can be found in a cell or cell extract after the cytoplasmic portion of a Newregulin-1 protein has been cleaved by a protease in the cell. See, for example, SEQ ID NO: 1. This protein lacks the amino terminal portion of Neu-1, preferably lacks at least 50,000 daltons of the extracellular region, and more preferably the entire extracellular region.fds In an embodiment, Nrg-1 CID includes at least 5, 10, 20 or more amino acids of the transmembrane region. Preferably the Nrg-ICD has a molecular weight of about 35,000 Daltons. The term “about” in this context means, as a minimum range, the normal variability encountered in an electrophoresis assay used for measureing protein size. Preferably the protein has an absolute molecular weight of between 25,000 daltons and 45,000 daltons. More preferably the protein has an absolute molecular weight of between 30,000 daltons and 40,000 daltons. In one embodiment, the protein is produced from Neuregulin-1 in cells such as 293T or 293S cells by one or more proteases present in these cells.

In another embodiment the Nrg-ICD has a molecular weight of about 53,000 daltons, preferably between 43,000 to 63,000 daltons and more preferably between 48,000 to 58,000 daltons. In one embodiment the protein is produced from Neuregulin-1 by proteolytic action.

The term “Eos” refers to a protein known by that name and all functional equivalents. For example, see “Eos: a novel member of the Ikaros gene family expressed predominantly in the developing nervous system” by Honma et al (FEBS Letters 447: 76-80, 1999.

The term “PDS-95” refers to a protein that is said to help build the physical scaffolding of the synapse that cells use to transmit the chemical messenger, or neurotransmitter, known as glutamate, to a target cell. The protein also apparently matures other aspects of the synapse—enhancing the clustering of glutamate receptors on the target cells receiving the chemical messenger, increasing the number and size of the dendritic spines that hold glutamate receptors, and increasing the number of glutamate neurotransmitters emitted from the releasing cell. This protein has been described by David S. Bredt et al (Science, Nov. 17, 2000 edition).

### IV. OTHER DEFINITIONS

To facilitate review of the embodiments and to provide greater clarity to the claims the following definitions for often-used terms are provided, which are consistent with usage in the field.
“Neuregulin-1 gene” (also “Neg-1 gene”) is a gene described herein, the extreme mutant forms of which are associated with neurological disease states. This definition is understood to include the various sequence polymorphisms that exist, wherein nucleotide substitutions in the gene sequence may affect the functioning of the gene product. This term relates primarily to an isolated coding sequence, but also can include some or all of the flanking regulatory elements and/or intron sequences.
“Nrg-1protein” is a protein encoded by the human Nrg-1 cDNA. This definition is understood to include the various sequence polymorphisms that exist, wherein amino acid substitutions in the protein sequence do not completely abolish the functions of the protein.
“cDNA” refers to complementary DNA, which lacks internal, non-coding segments (introns) and lacks regulatory sequences which determine transcription. cDNA is synthesized in the laboratory by reverse transcription from messenger RNA extracted from cells.
“DNA” refers to deoxyribonucleic acid. DNA is a long chain polymer which comprises the genetic material of most living organisms (some viruses have genes comprising ribonucleic acid). The repeating units in DNA polymers are four different nucleotides, each of which comprises one of the four bases, adenine, guanine, cytosine and thymine bound to a deoxyribose sugar to which a phosphate group is attached. Triplets of nucleotides, referred to as codons, in DNA molecules code for amino acids that are linked in a polypeptide. The term codon also is used for the corresponding (and complementary) sequences of three nucleotides in the mRNA into which the DNA sequence is transcribed. A standard nomenclature for DNA bases is employed.
“Nrg-1 Effector” refers to a molecule that has an effect on the Nrg-1 signaling pathway, and includes without limitation, Nrg-1 agonists, Nrg-1 antagonists, intracellular factors that interact with Nrg-1, intrinsic membrane proteins that interact with Nrg-1, extrinsic membrane proteins that interact with Nrg-1 and pharmaceuticals that are found to interact with any of the parts of the Neuregulin-1 signaling pathway.
“Homology” The percent of homology between two sequences is a function of the number of matching or homologous positions shared by the two sequences divided by the number of positions compared times 100. For example, if 6 of 10, of the positions in two sequences are matched or homologous then the two sequences are 60% homologous. By way of example, the DNA sequences ATTGCC and TATGGC share 50% homology. Generally, a comparison is made when two sequences are aligned to give maximum homology.
“Hybridizations” preferably are performed under stringent conditions, for example, at a salt concentration of no more than 1M and a temperature of at least 25 degrees C. For example, a condition of 5×SSPE (750 mM NaCl, 50 mM NaPhosphate, 5 mM EDTA, pH 7.4) and a temperature of 25-30 degrees C. is suitable for allele-specific probe hybridizations.
“Hybridization probes” are oligonucleotides capable of binding in a base-specific manner to a complementary strand of nucleic acid. Such probes include peptide nucleic acids, as described in Nielsen et al., Science 254, 1497-1500 (1991).
“Isolated” means that that the material has been removed from its original environment. For example, a naturally occurring DNA molecule present in a living animal is not isolated, but the same DNA molecule, separated from some or all of the coexisting materials in the natural system, is isolated.
An “isolated nucleic acid” refers to the predominant species present. That is, on a molar basis the isolated nucleic acid it is more abundant than any other individual species in the composition. Preferably, an isolated nucleic acid comprises at least about 50, 80 or 90 percent (on a molar basis) of all macromolecular species present. Most preferably, the object species is purified to “essential homogeneity” namely, that contaminant species cannot be detected in the composition by conventional detection methods.
“ORF” means open reading frame, which contains a series of nucleotide triplets (codons) coding for amino acids without any termination codons. These sequences are usually translatable into protein.
A “pathway gene” encodes a product, which may be regulatory RNA or a protein that is associated with the Nrg-1 gene product or with the biochemical pathways that extend to (for example, upstream signals) or from (for example, downstream signals) the Nrg-1 gene product.
“PCR” means polymerase chain reaction. This describes a technique in which cycles of denaturation, annealing with primer, and then extension with DNA polymerase are used to amplify the number of copies of a target DNA sequence.
“Polymorphism” refers to the occurrence of two or more genetically determined alternative sequences or alleles in a population. A polymorphic marker or site is the locus at which divergence occurs. Preferred markers have at least two alleles, each occurring at frequency of greater than 1%, and more preferably greater than 10% or 20% of a selected population. A polymorphic locus may be as small as one base pair. The first identified allelic form may be designated arbitrarily as a reference and other allelic forms designated as alternative or variant alleles. The allelic form occurring most frequently in a selected population is sometimes referred to as the wildtype form. Diploid organisms may be homozygous or heterozygous for allelic forms. A diallelic polymorphism has two forms. A triallelic polymorphism has three forms.
“Primer” refers to a single-stranded oligonucleotide capable of acting as a point of initiation of template-directed DNA synthesis under appropriate conditions (i.e., in the presence of four different nucleoside triphosphates and an agent for polymerization, such as, DNA or RNA polymerase or reverse transcriptase) in an appropriate buffer and at a suitable temperature. The appropriate length of a primer depends on the intended use of the primer but typically ranges from 15 to 30 nucleotides. Short primer molecules generally require cooler temperatures to form sufficiently stable hybrid complexes with the template. A primer need not reflect the exact sequence of the template but must be sufficiently complementary to hybridize with a template. The term primer site refers to the area of the target DNA to which a primer hybridizes. The term primer pair means a set of primers including a 5′ upstream primer that hybridizes with the 5′ end of the DNA sequence to be amplified and a 3′, downstream primer that hybridizes with the complement of the 3′ end of the sequence to be amplified.
The term “purified” does not necessitate absolute purity but relative purity. A reference to a protein as “purified” means that the protein is more pure with respect to the same protein in its natural environment within a cell or extracellular space from which it was purified.
A “reporter gene” encodes a sequence that produces a detectable substance such as mRNA, protein, or the activity of another reporter molecule encoded by the reporter gene. Examples of reporter gene sequences include the sequences encoding the enzymes beta.-galactosidase and luciferase.
A “reporter molecule”, as defined herein, is a molecule or atom which, by its chemical nature, provides an identifiable signal allowing detection. A reporter molecule may be encoded by a reporter gene. Detection can be either qualitative or quantitative. The present invention contemplates using any commonly used reporter molecules including radionucleotides, enzymes, biotin, psoralens, fluorophores, chelated heavy metals, and luciferin. The most commonly used reporter molecules are either enzymes, fluorophores, or radionucleotides linked to molecules to be detected. Commonly used enzymes include horseradish peroxidase, alkaline phosphatase, glucose oxidase and .alpha.-galactosidase, among others. Substrates for use with an enzyme may be chosen based on their ability to form a detectable colored product by the enzyme acting upon the substrate. For example, p-nitrophenyl phosphate is suitable for use with alkaline phosphatase conjugates; for horseradish peroxidase, 1.2-phenylenediamine, 5-aminosalicylic acid or toluidine are commonly used. The methods of using hybridization probes and protein conjugates that contain such labels are well known.

A “single nucleotide polymorphism” usually arises due to substitution of one nucleotide for another at the polymorphic site. A transition is the replacement of one purine by another purine or one pyrimidine by another pyrimidine. A transversion is the replacement of a purine by a pyrimidine or vice versa. Single nucleotide polymorphisms also can arise from a deletion of a nucleotide or an insertion of a nucleotide relative to a reference allele.

Additional definitions of specific terms used in receptor signaling biology are, for example, found in Mao et al., Molecular Cell, Vol. 7, 801-809 (2001) and common terms used in molecular biology may be found in Lewin, B. “Genes IV” published by Oxford University Press.

V. “Binding” Type Screening Methods

In embodiments of the invention, test substances such as peptides and synthetic chemicals are screened for their ability to interact with one or more components of the Nrg-1 system. The screen may test for binding to Nrg-1 or Eos directly, such as to an extracellular site on the Nrg-1 protein, to an intracellular site on the Nrg-1 protein, or to Eos. In a typical screen, a test substance is incubated with a specific target of the Nrg-1 system and binding is detected. The term “binding” in this context means that the association (determined by measuring an assocation constant) between test substance and the Nrg-1 component is greater than the association between the same test substance and other protein generally. Preferably the binding between test substance and Nrg-1 component is at least 10 times as strong and more preferably is at least 100 times as strong. Human serum albumin is preferred as a reference “other protein” because a large proportion of extracellular fluid protein is serum albumin.

In one embodiment a useful compound for screening preferably is a natural product, peptide, or other small compound capable of mimicking the effects of a naturally occuring regulator of the Neuregulin-1 system. One preferred screening method uses upregulation of PSD-95 or other gene activator, and another uses downstream signaling pathway molecules based on the above identified reactive binding partners. The screening methods may utilize Nrg-1, the PDS-95 target gene or use other related promoters to identify Nrg-1 inducers.

A particularly desirable screening test is carried out by incubating one or more ligand binding sites (typically provided as a peptide, fusion protein, Nrg-1 protein fragment or intact Nrg-1) in an aqueous solution with one or more test substances, and then determining binding between the test substance(s) and the ligand binding site. Binding assays are well known to the skilled artisan. For example, U.S. Pat. Nos. 5,976,814, 5,990,128 and 5,786,155 describe methods and tools for determining ligand binding, drug screening and associated techniques that are in use, and specifically are incorporated by reference in their entireties. In one embodiment, as explained in these patent documents, binding between a receptor expressed in intact cells and a test substance is used for screening.

In a preferred embodiment of testing and drug screening, a putative drug (chemical or protein) is desired that can bind to the intracellular portion of Nrg-1 protein, can bind Eos and facilitate nuclear translocation of Nrg-ICD, or that facilitates induction of PSD-95 synthesis in response to Nrg-ICD. Other types of compounds and proteins also may be detected by screening. Still others may be screened by assays that detect other biochemical behavior such as induction of synthesis of the Nrg-1 protein. In another screening methodology embodiment an intact cell that expresses the Nrg-1 protein is used and a biochemical or morphological event such as an enzyme reaction coupled to light production is detected to indicate that a test compound facilitates the ability of Nrg-1 to upregulate the PSD-95 gene.

A ligand that demonstrates specific binding in such a binding assay described here will, according to many embodiments, modulate the transcription of PSD-95. Specific Nrg-1 effectors may be discovered this way and used as pharmaceuticals to increase synapse morphology, with concomitant effects on memory and learning, in many instances. Other ligands of course can be discovered and are useful to modulate formation of PSD-95 by affecting the Nrg-1 regulatory system in a different direction.

In an embodiment, a compound for screening may be a natural product, peptide, or other small compound capable of mimicking the effects of a naturally occuring regulator that stimulates the production of Nrg-ICD or the ability of Nrg-ICD to upregulate PSD-95. In one embodiment a DNA encoding a portion of a sequence that emulates or that binds to a biologically effective portion of the Nrg-ICD, such as a site for proteolysis or for binding to Eos is obtained from a cDNA library prepared from tissue believed to possess the Neuregulon-1 polypeptide mRNA and that can express the DNA at a detectable level. For example, Neuregulon-1 polypeptide DNA can be conveniently obtained from a cDNA library prepared from fetal brain. The polypeptide-encoding gene may also be obtained from a genomic library or by oligonucleotide synthesis. Libraries are screened with probes (such as antibodies to the Nrg-ICD polypeptide or Eos polypeptide, or oligonucleotides of about 20-80 bases) designed to identify the gene of interest or the protein encoded by it. Screening the cDNA or genomic library with the selected probe may be conducted using standard procedures as described in chapters 10-12 of Sambrook et al., Molecular Cloning: A Laboratory Manual (New York: Cold Spring Harbor Laboratory Press, 1989). An alternative means to isolate the gene encoding Wnt polypeptide is to use PCR methodology as described in section 14 of Sambrook et al.

Amino acid sequence variants of polypeptide useful as Nrg-1 effectors according to this embodiment can be prepared by introducing appropriate nucleotide changes into the polypeptide DNA, or by synthesis of the desired polypeptide. Such variants represent insertions, substitutions, and/or specified deletions of, residues within or at one or both of the ends of the amino acid sequence of a naturally occurring human polypeptide. Preferably, these variants represent insertions and/or substitutions within or at one or both ends of the mature sequence, and/or insertions, substitutions and/or specified deletions within or at one or both of the ends of the signal sequence of the polypeptide. Any combination of insertion, substitution, and/or specified deletion is made to arrive at the final construct, provided that the final construct possesses the desired biological activity as defined herein. The amino acid changes may include post-translational processing of the polypeptide. Such processing may involve changing the number or position of glycosylation sites, altering the membrane anchoring characteristics, and/or altering the intracellular location of the polypeptide by inserting, deleting, or otherwise affecting the leader sequence of the polypeptide.

Variations in the native sequence as described above can be made using any of the techniques and guidelines for conservative and non-conservative mutations as for example set forth in U.S. Pat. No. 5,364,934. These include oligonucleotide-mediated (site-directed) mutagenesis, alanine scanning, and PCR mutagenesis. The nucleic acid (e.g., cDNA or genomic DNA) encoding the Wnt polypeptide is inserted into a replicable vector for further cloning (amplification of the DNA) or for expression. Many vectors are available for this use. The vector components generally include, but are not limited to, one or more of the following: a signal sequence, an origin of replication, one or more marker genes, an enhancer element, a promoter, and a transcription termination sequence.

This same general scheme may be used for discovery and modification of other polypeptides that resemble natural components of the Nrg-1 system as discovered and described herein. In many embodiments, however, pharmaceutical lead compounds are screened for their abilities to bind directly to the extracellular region of Nrg-1, intracellular region of Neuregulin-1 or to Eos. Such specific binding is particularly desirable for pharmacological intervention to promote improved synapse functioning because of decreased side effects from interacting with a specific (i.e. Nrg-1) receptor.

In one desirable embodiment a compound is selected for its effect on bone metabolism by looking for preferential binding to Nrg-ICD-Eos complex over Nrg-ICD alone. This differential binding is very useful for drug screening because such binding is expected to stabilize the complex and facilitate its formation. By choosing a lead compound that binds preferably to the complex, non-specific side effects of the pharmaceutical may be minimized. The phrase “that binds preferentially to” in this context means that the binding is at least 2 times, preferably at least 5 times, more preferably at least 20 times, yet more preferably at least 100 times and even more preferably more than 500 times as strong as determined by measuring and calculating an association constant at pH 7.0 at 37 degree C. in aqueous solution or suspension.

VI. Genetic Therapy

In a preferred embodiment, long term control of PSD-95 protein is achieved by transgenic expression of one or more of Neuregulin-1, Eos or of a factor that affects the interaction of these proteins to form a transcription modulator. A wide range of methods are useful for transgenic expression, including, for example, introduction of nucleic acid by a virus and introduction by vesicles. Preferably the transgenic expression occurs primarily in nerve tissue. This is best carried out by administration to nervous tissue or by selectively activating gene in nervous tissue compared with other tissue.

In another desirable embodiment an extracellular compound alters PSD-95 expression by interfering with binding between Nrg-ICD and Eos. In a preferred embodiment a polynucleic acid encoding a gene for a naturally occuring antagonist of this interaction is added in an antisense format such that an antisense strand forms and binds to messenger RNA, thus blocking translation and/or triggering nuclease destruction of the RNA. Recombination using antisense technology has previously been used to inhibit expression of specific gene products in mammalian cell lines (Kasid et al., Science 243:1354-1356 1989; Khoka et al., Science 243:947-950 1989; Izant et al., Science 229:345-352 1985) including some retroviruses; (von Ruden et al., J. Virol. 63:677-682 1989; and Chang et al., J. Virol. 61:921-924 1987). Each of these methods, and later developed methods specifically are contemplated for this embodiment of the invention.

VII. Dosage, Composition and Formulation of Pharmaceuticals for Prophylaxis and Therapy

Pharmaceutical compositions contemplated for embodiments of the invention comprise ingredients in an “effective amount” to achieve increased synaptic activity. Determination of the effective amounts is well within the capability of those skilled in the art, especially in light of the detailed disclosure provided herein. A therapeutically effective dose refers to that amount of the compound that results in amelioration of symptoms or a measurable increase in synaptic activity.

Generally speaking, an “effective amount” of a composition is that amount which produces a statistically significant effect. When determined by effects on stimulating the formation of PSD-95 in nerve cells in vitro, it is generally desirable to produce an increase in PSD-95 of at least 10%, 25% or even more than 50%, as compared to cells grown in the absence of added effector. For therapeutic uses, an “effective amount” is the amount of the composition comprising the effector required to provide a clinically significant increase in a measurable function such as freedom from tremors, memory ability and the like. Such amounts will depend, in part, on the particular condition to be treated and other factors evident to those skilled in the art. For example in memory function, an improvement is manifested as a statistically significant difference using a test. This can be seen as, for example, a 10-20% or more increase a test response. General guidance for treatment regimens is obtained from experiments carried out in animal models of the disease of interest.

Toxicity and therapeutic efficacy of such compounds also may be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio between LD50 and ED50. Compounds which exhibit high therapeutic indices are preferred. The data obtained from these cell culture assays and animal studies can be used in formulating a range of dosage for use in human. The dosage of such compounds lies preferably within a range of circulating concentrations that include the ED50 with little or no toxicity. The dosage may vary within this range depending upon the dosage form employed and the route of administration utilized. The exact formulation, route of administration and dosage can be chosen by the individual physician in view of the patient's condition. (See e.g. Fingl et al., 1975, in “The Pharmacological Basis of Therapeutics”, Ch. 1 p 1). Dosage amount and interval may be adjusted individually to provide plasma levels of the active moiety which are sufficient to maintain the desired effects.

In cases of local administration or selective uptake, the effective local concentration of the drug might not be related to plasma concentration. The amount of composition administered will, of course, be dependent on the subject being treated, on the subject's weight, the severity of the affliction, the manner of administration and the judgment of the prescribing physician. The pharmaceutical compositions of the present invention may be manufactured in a manner that is itself known, e.g., by means of conventional mixing, dissolving, granulating, dragee-making, levigating, emulsifying, encapsulating, entrapping or lyophilizing processes. Pharmaceutical compositions for use in accordance with the present invention thus may be formulated in conventional manner using one or more physiologically acceptable carriers comprising excipients and auxiliaries which facilitate processing of the active compounds into preparations which can be used pharmaceutically. Proper formulation is dependent upon the route of administration chosen.

For injection, the agent(s) of embodiments may be formulated in aqueous solutions, preferably in physiologically compatible buffers such as Hanks's solution, Ringer's solution, or physiological saline buffer. For transmucosal administration, penetrants appropriate to the barrier to be permeated are used in the formulation. Such penetrants are generally known in the art. For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration. For buccal administration, the compositions may take the form of tablets or lozenges formulated in conventional manner. For administration by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multidose containers, with an added preservative. The compositions may take such forms as suspensions, solutions or emulsions in oily or aqueous vehicles, and may contain formulatory agents such as suspending, stabilizing and/or dispersing agents. Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active ingredient may be in powder form for constitution with a suitable vehicle, e.g., sterile pyrogen-free water, before use. The compounds may also be formulated in rectal compositions such as suppositories or retention enemas, e.g., containing conventional suppository bases such as cocoa butter or other glycerides. In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

A preferred pharmaceutical carrier for the hydrophobic compounds of the invention is a cosolvent system comprising benzyl alcohol, a nonpolar surfactant, a water-miscible organic polymer, and an aqueous phase. Naturally, the proportions of a co-solvent system may be varied considerably without destroying its solubility and toxicity characteristics. Furthermore, the identity of the co-solvent components may be varied. Alternatively, other delivery systems for hydrophobic pharmaceutical compounds may be employed. Liposomes and emulsions are well known examples of delivery vehicles or carriers for hydrophobic drugs. Certain organic solvents such as dimethylsulfoxide also may be employed, although usually at the cost of greater toxicity. Additionally, the compounds may be delivered using a sustained-release system, such as semipermeable matrices of solid hydrophobic polymers containing the therapeutic agent. Various of sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols. Many of the compounds of the invention may be provided as salts with pharmaceutically compatible counterions. Pharmaceutically compatible salts may be formed with many acids, including but not limited to hydrochloric, sulfuric, acetic, lactic, tartaric, malic, succinic, etc. Salts tend to be more soluble in aqueous or other protonic solvents that are the corresponding free base forms.

Suitable routes of administration may, for example, include oral, rectal, transmucosal, transdermal, or intestinal administration; parenteral delivery, including intramuscular, subcutaneous, intramedullary injections, as well as intrathecal, direct intraventricular, intravenous, intraperitoneal, intranasal, or intraocular injections. Alternately, one may administer the compound in a local rather than systemic manner, for example, via injection of the compound directly into an affected area, often in a depot or sustained release formulation. Furthermore, one may administer the drug in a targeted drug delivery system, for example, in a liposome coated with an antibody specific for affected cells. The liposomes will be targeted to and taken up selectively by the cells.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. Compositions comprising a compound of the invention formulated in a compatible pharmaceutical carrier may also be prepared, placed in an appropriate container, and labelled for treatment of an indicated condition.

VIII. DNA-Based Diagnoses

One embodiment of the invention is the application of Nrg-1 DNA sequence information presented herein for genetic testing, carrier detection and prenatal diagnosis for genetic predisposition to weak synaptic function, hypersensitive synaptic functioning or other phenomena. Individuals carrying mutations in or near (within 5, 10, 20, 35, 50 or even more than 75 bases of) the peptide cleavage site, DNA binding site, Eos binding site or one of the nuclear binding sites of the Nrg-1 gene may be detected at the DNA level using any of a variety of techniques. For such a diagnostic procedure, a biological sample of the subject, which biological sample contains either DNA or RNA derived from the subject, is assayed for the presence of a mutant Nrg-1 gene. Suitable biological samples include samples containing genomic DNA or RNA obtained from body cells, such as those present in peripheral blood, urine, saliva, tissue biopsy, surgical specimen, amniocentesis samples and autopsy material. The detection in the biological sample of either a mutant Nrg-1 gene or a mutant Nrg-1 RNA may be performed by a number of methodologies, as outlined below.

A preferred embodiment of such detection techniques is the polymerase chain reaction amplification of reverse transcribed RNA (RT-PCR) of RNA isolated from bone marrow cells or other cells followed by direct DNA sequence determination of the products. The presence of one or more nucleotide difference between the obtained sequence and a consensus DNA sequence, and especially, differences in the portion of the nucleotide sequence that encode the region that binds to Eos, are taken as indicative of a potential Nrg-1 gene mutation.

The detection of specific DNA mutations may be achieved by methods such as hybridization using specific oligonucleotides (Wallace et al. (1986). Cold Spring Harbor Symp. Quant. Biol. 51:257-261), direct DNA sequencing (Church and Gilbert (1988). Proc. Natl. Acad. Sci. USA 81:1991-1995), the use of restriction enzymes (Flavell et al. (1978). Cell 15:25), discrimination on the basis of electrophoretic mobility in gels with denaturing reagent (Myers and Maniatis (1986). Cold Spring Harbor Symp. Quant. Biol. 51:275-284), RNase protection (Myers et al. (1985). Science 230:1242), chemical cleavage (Cotton et al. (1985). Proc. Natl. Acad. Sci. USA 85:43974401), and the ligase-mediated detection procedure (Landegren et al., 1988).

Oligonucleotides specific to normal or mutant sequences are chemically synthesized using commercially available machines, labelled radioactively with isotopes (such as ³²P) or non-radioactively (with tags such as biotin (Ward and Langer et al. (1981). Proc. Natl. Acad. Sci. USA 78:6633-6657), and hybridized to individual DNA samples immobilized on membranes or other solid supports by dot-blot or transfer from gels after electrophoresis. The presence or absence of these specific sequences are determined optically by methods such as autoradiography or fluorometric (Landegren, et al., 1989) or calorimetric reactions (Gebeyehu et al. (1987). Nucleic Acids Res. 15:4513-4534).

Sequence differences between normal and mutant functional sites of the gene also may be revealed by the direct DNA sequencing method of Church and Gilbert (1988). Cloned DNA segments may be used as probes to detect specific DNA segments. The sensitivity of this method is greatly enhanced when combined with PCR (Wrichnik et al., 1987; Wong et al., 1987; Stoflet et al. (1988). Science 239:491-494). In this approach, a sequencing primer which lies within the amplified sequence is used with double-stranded PCR product or single-stranded template generated by a modified PCR. The sequence determination is performed by conventional procedures with radiolabeled nucleotides or by automatic sequencing procedures with fluorescent tags.

Sequence alterations occasionally may generate fortuitous restriction enzyme recognition sites or may eliminate existing restriction sites. Changes in restriction sites are revealed by the use of appropriate enzyme digestion followed by conventional gel-blot hybridization (Southern (1975). J. Mol. Biol. 98:503). DNA fragments carrying the site (either normal or mutant) are detected by their reduction in size or increase of corresponding restriction fragment numbers. Genomic DNA samples may also be amplified by PCR prior to treatment with the appropriate restriction enzyme; fragments of different sizes are then visualized under UV light in the presence of ethidium bromide after gel electrophoresis.

Genetic testing based on DNA sequence differences may be achieved by detection of alteration in electrophoretic mobility of DNA fragments in gels with or without denaturing reagent. Small sequence deletions and insertions can be visualized by high-resolution gel electrophoresis. For example, a PCR product with small deletions is clearly distinguishable from a normal sequence on an 8% non-denaturing polyacrylamide gel (WO 91/10734, Nagamine et al. Am. J. Hum. Genet. 45:337-339 (1989). DNA fragments of different sequence compositions may be distinguished on denaturing formamide gradient gels in which the mobilities of different DNA fragments are retarded in the gel at different positions according to their specific “partial-melting” temperatures (Myers et al. (1985). Science 230:1242). Alternatively, a method of detecting a mutation comprising a single base substitution or other small change could be based on differential primer length in a PCR. For example, an invariant primer could be used in addition to a primer specific for a mutation. The PCR products of the normal and mutant genes can then be differentially detected in acrylamide gels.

In addition to conventional gel-electrophoresis and blot-hybridization methods, DNA fragments may also be visualized by methods where the individual DNA samples are not immobilized on membranes. The probe and target sequences may be both in solution, or the probe sequence may be immobilized (Saiki et al. Proc. Nat. Acad. Sci. USA 86:6230-6234 (1989). A variety of detection methods, such as autoradiography involving radioisotopes, direct detection of radioactive decay (in the presence or absence of scintillant), spectrophotometry involving calorigenic reactions and fluorometry involved fluorogenic reactions, may be used to identify specific individual genotypes.

Other more recent methods to detect genetic polymorphism are useful for embodiments of the invention. A particularly good example is that disclosed in U.S. Pat. No. 5,856,104 issued to Chen et al. Jan. 5, 1999, the contents of which specifically are incorporated by reference in its entirety.

If more than one mutation is frequently encountered in the Nrg-1 gene, a system capable of detecting such multiple mutations would be desirable. For example, a PCR with multiple, specific oligonucleotide primers and hybridization probes may be used to identify all possible mutations at the same time (Chamberlain et al. (1988). Nucl. Acids Res. 16:1141-1155 (1988)). The procedure may involve immobilized sequence-specific oligonucleotides probes (Saiki et al. Proc. Nat. Acad. Sci. USA 86:6230-6234) (1989).

IX. DNA Vectors Useful for Improved Synaptic Function

Other embodiments of the invention provide recombinant DNA vectors comprising the desirable DNA sequences that affect synapse function and other functions.

With the provision of the Nrg-1 cDNA and/or Eos cDNAs and the understanding of the role and mechanisms of the coded proteins in the Neuregulin-1 control system, vectors that contain Nrg-1 DNA may be used for diagnosis and therapy. Furthermore, the DNA sequence of the Nrg-1 cDNA and polymorphic or mutated Nrg-1 cDNAs isolated from patients can be manipulated in studies to further understand the expression of the gene and the function of its product. In this way, further ligands usful for embodiments of the invention and mechanisms involved in control of synapse plasticity and related biological functions can be discovered.

Mutant versions of Nrg-1 DNA and Eos DNA at specific sites of proteolytic cleavage, nuclear transport signals, Eos binding, Nrg-1 binding, and DNA binding, and other nucleic acid sequences may be isolated based upon information contained herein. This information may be used to detect alteration in expression patterns in terms of relative quantities, tissue specificity and functional properties of the encoded Nrg-1 protein in individuals to determine their PSD-95 induction status. Partial or full-length DNA sequences, which encode Nrg-1 may be ligated into bacterial expression vectors. Methods for expressing large amounts of protein from a cloned gene that is introduced into Escherichia coli (E. coli) may be utilized to purify, localize and study the function of proteins involved in the regulation. For example, fusion proteins consisting of amino terminal peptides encoded by a portion of the E. coli lacZ or trpE gene linked to Nrg-1 proteins may be used to prepare polyclonal and monoclonal antibodies against these proteins. Thereafter, these antibodies may be used to purify proteins by immunoaffinity chromatography, in diagnostic assays to quantitate the levels of protein and to localize proteins in tissues and individual cells by immunofluorescence.

Intact native protein also may be produced in E. coli in large amounts for functional studies. Native proteins can be produced in bacteria by placing a strong, regulated promoter and an efficient ribosome binding site upstream of the cloned gene. If low levels of protein are expressed, additional steps may be taken to increase protein production; if high levels of protein are expressed, purification is easier. Suitable methods are presented in Sambrook et al. (1989). In Molecular Cloning: A Laboratory Manual, Cold Spring Harbor, N.Y. and are well known in the art. Often, proteins expressed at high levels are found in insoluble inclusion bodies. Methods for extracting proteins from these aggregates are described by Sambrook et al. (1989). In Molecular Cloning: A Laboratory Manual, Cold Spring Harbor, N.Y. Vector systems suitable for the expression of lacZ fusion genes include the pUR series of vectors (Ruther and Muller-Hill (1983). EMBO J. 2:1791), pEXl-3 (Stanley and Luzio (1984). EMBO J.3:1429) and pMR100 (Gray et al. (1982). Proc. Natl. Acad. Sci. USA 79:6598). Vectors suitable for the production of intact native proteins include pKC30 (Shimatake and Rosenberg (1981). Nature (London) 292:128), pKK177-3 (Amann and Brosius (1985). Gene 40:183.) and pET-3 (Studiar and Moffatt (1986). J. Mol. Biol. 189:113).

Methods and plasmid vectors for producing fusion proteins and intact native proteins in bacteria are described in Sambrook et al. (1989). In Molecular Cloning: A Laboratory Manual, Cold Spring Harbor, N.Y. (ch. 17, herein incorporated by reference). Such fusion proteins may be made in large amounts, are easy to purify, and can be used to elicit antibody response. Nrg-1 fusion proteins may be isolated from protein gels, lyophilized, ground into a powder and used as an antigen. The DNA sequence can also be transferred from its existing context in pREP4 to other cloning vehicles, such as other plasmids, bacteriophages, cosmids, animal viruses and yeast artificial chromosomes (YACs) (Burke et al. (1987). Science 236:806-812). These vectors may then be introduced into a variety of hosts including somatic cells, and simple or complex organisms, such as bacteria, fungi (Timberlake and Marshall (1989). Science 244:1313-1317), invertebrates, plants (Gasser and Fraley (1989). Science 244:1293), and pigs (Pursel et al. (1989). Science 244:1281-1288), which cell or organisms are rendered transgenic by the introduction of the heterologous Nrg-1 or Eos cDNA, or portions thereof.

For expression in mammalian cells, the cDNA sequence may be ligated to heterologous promoters, such as the simian virus (SV)40, promoter in the pSV2 vector (Mulligan and Berg, 1981), and introduced into cells, such as monkey COS-1 cells (Gluzman (1981). Cell 23:175-182), to achieve transient or long-term expression. The stable integration of the chimeric gene construct may be maintained in mammalian cells by biochemical selection, such as neomycin (Southern and Berg (1982). J. Mol. Appl. Genet. 1:327-341) and mycophoenolic acid (Mulligan and Berg, 1981).

DNA sequences can be manipulated with standard procedures such as restriction enzyme digestion, fill-in with DNA polymerase, deletion by exonuclease, extension by terminal deoxynucleotide transferase, ligation of synthetic or cloned DNA sequences, site-directed sequence-alteration via single-stranded bacteriophage intermediate or with the use of specific oligonucleotides in combination with PCR.

The cDNA sequence (or portions derived from it) or a mini gene (a cDNA with an intron and its own promoter) is introduced into eukaryotic expression vectors by conventional techniques. These vectors are designed to permit the transcription of the cDNA eukaryotic cells by providing regulatory sequences that initiate and enhance the transcription of the cDNA and ensure its proper splicing and polyadenylation. Vectors containing the promoter and enhancer regions of the SV40 or long terminal repeat (LTR) of the Rous Sarcoma virus and polyadenylation and splicing signal from SV40 are readily available (Mulligan et al., 1981; Gorman et al. (1982). Proc. Natl. Acad. Sci. USA 78:6777-6781). The level of expression of the cDNA can be manipulated with these types of vector, either by using promoters that have different activities (for example, the baculovirus pAC373 can express cDNAs at high levels in S. frugiperda cells (Summers and Smith (1985). In Genetically Altered Viruses and the Environment, Fields et al. (Eds.) 22:319-328, Cold Spring Harbor Laboratory Press, Cold Spring Harbor, N.Y.) or by using vectors that contain promoters amenable to modulation, for example, the glucocorticoid-responsive promoter from the mouse mammary tumor virus (Lee et al. (1982). Nature 294:228). The expression of the cDNA can be monitored in the recipient cells 24 to 72 hours after introduction (transient expression).

In addition, some vectors contain selectable markers such as the gpt (Mulligan and Berg, 1981) or neo (Southern and Berg (1982). J. Mol. Appl. Genet. 1:327-341) bacterial genes. These selectable markers permit selection of transfected cells that exhibit stable, long-term expression of the vectors (and therefore the cDNA). The vectors can be maintained in the cells as episomal, freely replicating entities by using regulatory elements of viruses such as papilloma (Schafner (1980). Proc. Natl. Acad. Sci. USA 77:2163-2167). Proc. Natl. Acad. Sci. USA 77:2163-2167). Mol. Cell Biol. 1:486) or Epstein-Barr (Sugden et al. (1985). Mol. Cell Biol. 5:410). Such episomal vectors are exemplified by the pREP4 Epstein-Barr virus vector in which the cDNA library described in Example 2 herein was constructed. Alternatively, one can also produce cell lines that have integrated the vector into genomic DNA. Both of these types of cell lines produce the gene product on a continuous basis. One can also produce cell lines that have amplified the number of copies of the vector (and therefore of the cDNA as well) to create cell lines that can produce high levels of the gene product (Alt et al. (1978). J. Biol. Chem. 253:1357).

The transfer of DNA into eukaryotic, in particular human or other mammalian cells, is now a conventional technique. The vectors are introduced into the recipient cells as pure DNA (transfection) by, for example, precipitation with calcium phosphate (Graham and vander Eb (1973). Virology 52:466) or strontium phosphate (Brash et al. (1987). Mol. Cell Biol. 7:2013), electroporation (Neumann et al. (1982). EMBO J 1:841), lipofection (Felgner et al. (1987). Proc. Natl. Acad. Sci. USA 84:7413), DEAE dextran (McCuthan et al. (1968). J. Natl. Cancer Inst. 41:351), microinjection (Mueller et al. (1978). Cell 15:579), protoplast fusion (Schafner (1980). Proc. Natl. Acad. Sci. USA 77:2163-2167), or pellet guns (Klein et al., 1987). Alternatively, the cDNA can be introduced by infection with virus vectors. Systems are developed that use, for example, retroviruses (Bernstein et al. (1985). Gen. Engr'g 7:235), adenoviruses (Ahmad et al. (1986). J. Virol. 57:267.), or Herpes virus (Spaete et al. (1982). Cell 30:295).

These eukaryotic expression systems can be used for studies of the Nrg-1 and Eos genes and mutant forms of these genes, the Nrg-1 and Eos proteins and mutant forms of these proteins. Such uses include, for example, the identification of regulatory elements located in the 5′ region of the Nrg-1 gene on genomic clones that can be isolated from human genomic DNA libraries using the information contained in the present invention. The eukaryotic expression systems may also be used to study the function of the normal complete protein, specific portions of the protein, or of naturally occurring or artificially produced mutant proteins. Naturally occurring mutant proteins exist in patients with Nrg-1, while artificially produced mutant proteins can be designed by site directed mutagenesis as described above. These latter studies may probe the function of any desired amino acid residue in the protein by mutating the nucleotide coding for that amino acid.

Using the above techniques, the expression vectors containing the Nrg-1 gene sequence or fragments or variants or mutants thereof can be introduced into human cells, mammalian cells from other species or non-mammalian cells as desired. The choice of cell is determined by the purpose of the treatment. Generally, transfer into cells that forms synapses is preferred.

The following is provided as one exemplary method to express Nrg-1 polypeptide from the cloned Nrg-1 cDNA sequences in mammalian cells. Cloning vector pXTI, commercially available from Stratagene, contains the Long Terminal Repeats (LTRs) and a portion of the GAG gene from Moloney Murine Leukemia Virus. The position of the viral LTRs allows highly efficient, stable transfection of the region within the LTRs. The vector also contains the Herpes Simplex Thymidine Kinase promoter (TK), active in embryohal cells and in a wide variety of tissues in mice, and a selectable neomycin gene conferring G418 resistance. Two unique restriction sites BglII and XhoI are directly downstream from the TK promoter. Nrg-1 cDNA, including the entire open reading frame for the Nrg-1 protein and the 3′ untranslated region of the cDNA is cloned into one of the two unique restriction sites downstream from the promoter.

The ligated product is transfected into mouse NIH 3T3 cells using Lipofectin (Life Technologies, Inc.) under conditions outlined in the product specification. Positive transfectants are selected after growing the transfected cells in 600.mu.g/ml G418 (Sigma, St. Louis, Mo.). The protein is released into the supernatant and may be purified by standard immunoaffinity chromatography techniques using antibodies raised against the Nrg-1 protein, as described below.

Expression of the Nrg-1 protein in eukaryotic cells may also be used as a source of proteins to raise antibodies. The Nrg-1 protein may be extracted following release of the protein into the supernatant as described above, or, the cDNA sequence may be incorporated into a eukaryotic expression vector and expressed as a chimeric protein with, for example, beta-globin. Antibody to .beta.-globin is thereafter used to purify the chimeric protein. Corresponding protease cleavage sites engineered between the beta.-globin gene and the cDNA are then used to separate the two polypeptide fragments from one another after translation. One useful expression vector for generating .beta.-globin chimeric proteins is pSG5 (Stratagene). This vector encodes rabbit .beta.-globin.

The recombinant cloning vector, according to this invention, then comprises the selected DNA of the DNA sequences of this invention for expression in a suitable host. The DNA is operatively linked in the vector to an expression control sequence in the recombinant DNA molecule so that the Nrg-1 polypeptide can be expressed. The expression control sequence may be selected from the group consisting of sequences that control the expression of genes of prokaryotic or eukaryotic cells and their viruses and combinations thereof. The expression control sequence may be specifically selected from the group consisting of the lac system, the trp system, the tac system, the trc system, major operator and promoter regions of phage lambda, the control region of fd coat protein, the early and late promoters of SV40, promoters derived from polyoma, adenovirus, retrovirus, baculovirus and simian virus, the promoter for 3-phosphoglycerate kinase, the promoters of yeast acid phosphatase, the promoter of the yeast alpha-mating factors and combinations thereof.

Retroviruses have been considered the preferred vector for experiments in gene therapy, with a high efficiency of infection and stable integration and expression (Orkin et al. (1988). Prog. Med. Genet. 7:130). The full length Nrg-1 gene, optionally with the Eos gene, or DNA can be cloned into a retroviral vector and driven from either its endogenous promoter or from the retrovital LTR (long terminal repeat). Other viral transfection systems may also be utilized for this type of approach, including Adeno-Associated virus (AAV) (McLaughlin et al. (1988). J. Virol. 62:1963), Vaccinia virus (Moss et al. (1987). Annu. Rev. Immunol. 5:305), Bovine Papilloma virus (Rasmussen et al. (1987). Methods Enzymol. 139:642) or members of the herpesvirus group such as Epstein-Barr virus (Margolskee et al. (1988). Mol. Cell. Biol. 8:2837-2847).

The mouse is an extremely useful experimental organism for experiments with transgenic technology. The cloning of the mouse Nrg-1 homologue permits generation of a mouse model for Nrg-1 by targeted gene replacement in mouse embryonic stem cells (Sedivy and Joyner (1992). In Gene Targeting, W. H. Freeman and Company, New York). This in turn, will facilitate the study of the abnormal developmental processes leading to the pleiotropic phenotype of synaptic processes.

The host cell, which may be transfected with the vector of this invention, may be selected from the group consisting of E. coli, Pseudomonas, Bacillus subtilis, Bacillus stearothermophilus or other bacilli; other bacteria; yeast; fungi; insect; mouse or other animal; or plant hosts; or human tissue cells.

It is appreciated that for mutant or variant DNA sequences, similar systems are employed to express and produce the mutant product.

Diagnosis of PSD-9S Protein Based Synapse Function Using the Nrg-1 Gene Sequence

One embodiment of the invention is a screening method to determine if a subject carries a Nrg-1 gene with a mutation in a functional region and or to determine resultant altered synapse strength and activity. The method comprises the steps of: providing a biological sample obtained from the subject, which sample includes DNA or RNA, providing an assay for detecting in the biological sample the presence of at least one member from the group consisting of a mutant Nrg-1 gene and a mutant Nrg-1 RNA. A preferred embodiment of this method is described wherein the assay comprises a method selected from the group consisting of: hybridization with oligonucleotides; PCR amplification of the Nrg-1 gene or a part thereof using oligonucleotide primers; RT-PCR amplification of the Nrg-1 RNA or a part thereof using oligonucleotide primers, and direct sequencing of the Nrg-1 gene of the subject's genome using oligonucleotide primers. When the availability of intron sequence data from the splice sites of the human Nrg-1 gene and polymerase chain reactions for the amplification of these sequences from genomic DNA, as provided by this invention, will permit the analysis of these regions for potential splice site mutations. Furthermore, the efficiency of these molecular genetic methods should permit a more rapid classification of patients than is possible with the labor intensive method of classical complementation analysis.

A embodiment is a method for screening a subject to assay for the presence of a mutation in a functional site of a Nrg-1 gene, comprising the steps of: providing a biological sample of the subject which sample contains cellular proteins and providing an immunoassay for determining synapse stimulation quality of Nrg-1 protein in the biological sample, based on differential binding to antibodies. Most preferred is an embodiment that used monoclonal antibodies that react with ligand binding site(s) on the extracellular portion of the Nrg-1 protein. The generation of monoclonal antibodies using peptide, chimeric protein, or entire intact Nrg-1 protein uses routine methodology.

In further embodiments, a subject is screened to determine his or her polymorphism for the Nrg-1 gene. This information is used to predict propensity to alterations in PDS-95 and to synapse activity. Various techniques for assaying genetic polymorphism are known, as exemplified in U.S. Pat. Nos. 6,074,831; 5,364,759; 5,614,364; and 5,856,104, the contents of which specifically are incorporated by reference in their entireties, particularly with respect to their descriptions on how to detect polymorphism of a gene.

Another embodiment of the invention is an immunoassay for detecting predisposition induction of the PSD-95 gene, comprising antibodies that specifically bind Nrg-ICD protein, wherein the antibodies are selected from the group consisting of monoclonal antibodies and polyclonal antibodies. Techniques for using the antibodies in a kit (that is, in a package with an instructional chart or package insert) are routine and are contemplated as further embodiments of the invention.

Through the manipulation of the nucleotide sequence of the human or murine cDNAs provided by this invention by standard molecular biology techniques, variants of the proteins described herein may be made which differ in precise amino acid sequence from the disclosed proteins yet which maintain the essential characteristics of the proteins or which are selected to differ in some characteristics from these proteins. Such variants are another aspect of the present invention.

### Nucleic Acids Useful for Embodiments of the Invention

A large variety of isolated human and animal Nrg-1 and Eos cDNA sequences, and partial sequences that hybridize with these genes are useful for detecting an individual's tendency for PDS-95 regulation and synapse activity according to embodiments of the invention. Representative nucleic acid sequences in this context have been shown in the figures. Still further variants of these sequences and sequences of nucleic acids that hybridize with those shown in the figures also are comprehended for use in detection and alteration of PDS-95 status/tendency, as are the genomic genes from which these cDNAs are derived.

Many of the contemplated variant DNA molecules include those created by standard DNA mutagenesis techniques, for example, M13 primer mutagenesis. Details of these techniques are provided in Sambrook et al. (1989). In Molecular Cloning: A Laboratory Manual, Cold Spring Harbor, N.Y. By the use of such techniques, variants may be created which differ in minor ways from those disclosed. DNA molecules and nucleotide sequences which are derivatives of those specifically disclosed herein and which differ from those disclosed by the deletion, addition or substitution of nucleotides while still encoding a protein which possesses the functional characteristic of the Nrg-1 protein are comprehended by this invention. Also within the scope of this invention are small DNA molecules which are derived from the disclosed DNA molecules. Such small DNA molecules include oligonucleotides suitable for use as hybridization probes or polymerase chain reaction (PCR) primers. As such, these small DNA molecules will comprise at least a segment of an Nrg-1 cDNA molecule or the Nrg-1 gene and, for the purposes of PCR, will comprise at least a 10-15 nucleotide sequence and, more preferably, a 15-30 nucleotide sequence of the Nrg-1 cDNA or the Nrg-1 gene. DNA molecules and nucleotide sequences which are derived from the disclosed DNA molecules as described above may also be defined as DNA sequences which hybridize under stringent conditions to the DNA sequences disclosed, or fragments thereof.

Hybridization conditions corresponding to particular degrees of stringency vary depending upon the nature of the hybridization method of choice and the composition and length of the hybridizing DNA used. Generally, the temperature of hybridization and the ionic strength (especially the sodium ion concentration) of the hybridization buffer will determine the stringency of hybridization. Calculations regarding hybridization conditions required for attaining particular degrees of stringency are discussed by Sambrook et al. (1989). In Molecular Cloning: A Laboratory Manual, Cold Spring Harbor, N.Y., chapters 9 and 11, herein incorporated by reference. By way of illustration only, a hybridization experiment may be performed by hybridization of a DNA molecule (for example, a deviation of the Nrg-1 cDNA) to a target DNA molecule (for example, the Nrg-1 cDNA) which has been electrophoresed in an agarose gel and transferred to a nitrocellulose membrane by Southern blotting (Southern (1975). J. Mol. Biol. 98:503), a technique well known in the art and described in Sambrook et al. (1989). In Molecular Cloning: A Laboratory Manual, Cold Spring Harbor, N.Y. Hybridization with a target probe labeled with isotopic P (32) labelled-dCTP generally is carried out in a solution of high ionic strength such as 6 times SSC at a temperature that is 20-25 degrees Celsius, below the melting temperature, Tₘ, described below. For such Southern hybridization experiments where the target DNA molecule on the Southern blot contains 10 ng of DNA or more, hybridization is typically carried out for 6-8 hours using 1-2 ng/ml radiolabeled probe (of specific activity equal to 10⁹ CPM/mug or greater). Following hybridization, the nitrocellulose filter is washed to remove background hybridization. The washing conditions should be as stringent as possible to remove background hybridization but to retain a specific hybridization signal. The term “T_{m”} represents the temperature above which, under the prevailing ionic conditions, the radiolabeled probe molecule will not hybridize to its target DNA molecule. The Tₘ of such a hybrid molecule may be estimated from the following equation (Bolton and McCarthy (1962). Proc. Natl. Acad. Sci. USA 48:1390): Tₘ=81.5 degrees C.−16.6(log₁₀ of sodium ion concentration)+0.41(% G+C)−0.63(% formamide)−(600/l) where l=the length of the hybrid in base pairs. This equation is valid for concentrations of sodium ion in the range of 0.01M to 0.4M, and it is less accurate for calculations of Tₘ in solutions of higher sodium ion concentration. The equation also is valid for DNA having G+C contents within 30% to 75%, and also applies to hybrids greater than 100 nucleotides in length. The behavior of oligonucleotide probes is described in detail in Ch. 11 of Sambrook et al. (1989). In Molecular Cloning: A Laboratory Manual, Cold Spring Harbor, N.Y.

Thus, by way of example of a 150 base pair DNA probe derived from a section of 150 base pairs of the open reading frame of the Nrg-1 cDNA (with an assumed % GC=45%), a calculation of hybridization conditions required to give particular stringencies may be made as follows:

For this example, it is assumed that the filter will be washed in 0.3×SSC solution following hybridization, thereby sodium ion=0.045M; % GC=45%; Formamide concentration=0 1=150 base pairs (EQU1) and so Tₘ=74.4 degrees C.

The Tₘ of double-stranded DNA decreases by 1-1.5 degrees C with every 1% decrease in homology (Bonner et al. (1973). J. Mol. Biol. 81:123). Therefore, for this given example, washing the filter in 0.3 times SSC at 59.4-64.4 degrees C. will produce a stringency of hybridization equivalent to 90%; that is, DNA molecules with more than 10% sequence variation relative to the target Nrg-1 cDNA will not hybridize. Alternatively, washing the hybridized filter in 0.3 times SSC at a temperature of 65.4-68.4 degrees C. will yield a hybridization stringency of 94%; that is, DNA molecules with more than 6% sequence variation relative to the target Nrg-1 cDNA molecule will not hybridize. The above example is given entirely by way of theoretical illustration. One skilled in the art will appreciate that other hybridization techniques may be utilized and that variations in experimental conditions will necessitate alternative calculations for stringency.

In preferred embodiments of the present invention, stringent conditions may be defined as those under which DNA molecules with more than 25% sequence variation (also termed “mismatch”) will not hybridize. In a more preferred embodiment, stringent conditions are those under which DNA molecules with more than 15% mismatch will not hybridize, and more preferably still, stringent conditions are those under which DNA sequences with more than 10% mismatch will not hybridize. In a most preferred embodiment, stringent conditions are those under which DNA sequences with more than 6% mismatch will not hybridize.

The degeneracy of the genetic code further widens the scope of the present invention as it enables major variations in the nucleotide sequence of a DNA molecule while maintaining the amino acid sequence of the encoded protein. The nucleotide sequence of the Nrg-1 cDNA could be changed at various positions to other codons without affecting the amine acid composition of the encoded protein or the characteristics of the protein. The genetic code and variations in nucleotide codons for particular amine acids is presented in Tables 4 and 5. Based upon the degeneracy of the genetic code, variant DNA molecules may be derived from the cDNA molecules disclosed herein using standard DNA mutagenesis techniques as described above, or by synthesis of DNA sequences. DNA sequences which do not hybridize under stringent conditions to the cDNA sequences disclosed by virtue of sequence variation based on the degeneracy of the genetic code are included in an embodiment.

TABLE 3
The Genetic Code

| First | | Second | Third | | |
|---|---|---|---|---|---|
| Position | | Position | Position | | |
| (5′) | T | C | A | G | (3′) |
| T | Phe | Ser | Tyr | Cys | T |
| | Phe | Ser | Tyr | Cys | C |
| | Leu | Ser | Stop | Stop | A (oche) |
| | Leu | Ser | Stop (amb) | Trp | G |
| C | Leu | Pro | His | Arg | T |
| | Leu | Pro | His | Arg | C |
| | Leu | Pro | Gln | Arg | A |
| | Leu | Pro | Gln | Arg | G |
| A | Ile | Thr | Asn | Ser | T |
| | Ile | Thr | Asn | Ser | C |
| | Ile | Thr | Lys | Arg | A |
| | Met | Thr | Lys | Arg | G |
| G | Val | Ala | Asp | Gly | T |
| | Val | Ala | Asp | Gly | C |
| | Val | Ala | Glu | Gly | A |
| | Val (Met) | Ala | Glu | Gly | G |

“Stop (och)” stands for the ocre termination triplet, and “Stop (amb)” for the amber. ATG is the most common initiator codon; GTG usually codes for valine, but it can also code for methionine to initiate an mRNA chain.

TABLE 4
The Degeneracy of the Genetic Code

| Number of | | Total |
|---|---|---|
| Synonymous | | Number of |
| Codons | Amino Acid | Codons |
| 6 | Leu, Ser, Arg | 18 |
| 4 | Gly, Pro, Ala, Val, Thr | 20 |
| 3 | Ile | 3 |
| 2 | Phe, Tyr, Cys, His, Gln, | 18 |
| | Glu, Asn, Asp, Lys | |
| 1 | Met, Trp | 2 |

| | |
|---|---|
| Total number of codons for amino acids | 61 |
| Number of codons for termination | 3 |
| Total number of codons in genetic code | 64 |

One skilled in the art will recognize that the DNA mutagenesis techniques described above may be used not only to produce variant DNA molecules, but will also facilitate the production of proteins which differ in certain structural aspects from the Nrg-1 and Eos protein, yet which proteins are clearly derivative of this protein and which maintain the essential characteristics of the Nrg-1 and Eos proteins. Newly derived proteins may also be selected in order to obtain variations on the characteristic of the Nrg-1 and Eos proteins, as will be more fully described below. Such derivatives include those with variations in amino acid sequence including minor deletions, additions and substitutions.

While the site for introducing an amino acid sequence variation is predetermined, the mutation per se need not be predetermined. For example, in order to optimize the performance of a mutation at a given site, random mutagenesis may be conducted at the target codon or region and the expressed protein variants screened for the optimal combination of desired activity. Techniques for making substitution mutations at predetermined sites in DNA having a known sequence as described above are well known.

Amino acid substitutions are typically of single residues; insertions usually will be on the order of about from 1 to 10 amino acid residues; and deletions will range about from 1 to 30 residues. Deletions or insertions preferably are made in adjacent pairs, i.e., a deletion of 2 residues or insertion of 2 residues. Substitutions, deletions, insertions or any combination thereof may be combined to arrive at a final construct. Obviously, the mutations that are made in the DNA encoding the protein must not place the sequence out of reading frame and preferably will not create complementary regions that could produce secondary mRNA structure (EP 75,444A).

Substitutional variants are those in which at least one residue in the amino acid sequence has been removed and a different residue inserted in its place. Such substitutions generally are made in accordance with the following Table 5 when it is desired to finely modulate the characteristics of the protein. Table 5 shows amino acids which may be substituted for an original amino acid in a protein and which are regarded as conservative substitutions.

TABLE 5

| Original Residue | Conservative Substitutions |
|---|---|
| Ala | ser |
| Arg | lys |
| Asn | gln, his |
| Asp | glu |
| Cys | ser |
| Gln | asn |
| Glu | asp |
| Gly | pro |
| His | asn; gln |
| Ile | leu, val |
| Leu | ile; val |
| Lys | arg; gln; glu |
| Met | leu; ile |
| Phe | met; leu; tyr |
| Ser | thr |
| Thr | ser |
| Trp | tyr |
| Tyr | trp; phe |
| Val | ile; leu |

Substantial changes in function or immunological identity are made by selecting substitutions that are less conservative than those in Table 5, i.e., selecting residues that differ more significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. The substitutions which in general are expected to produce the greatest changes in protein properties will be those in which (a) a hydrophilic residue, e.g., seryl or threonyl, is substituted for (or by) a hydrophobic residue, e.g., leucyl, isoleucyl, phenylalanyl, valyl or alanyl; (b) a cysteine or proline is substituted for (or by) any other residue; (c) a residue having an electropositive side chain, e.g., lysyl, arginyl, or histadyl, is substituted for (or by) an electronegative residue, e.g., glutamyl or aspartyl; or (d) a residue having a bulky side chain, e.g., phenylalanine, is substituted for (or by) one not having a side chain, e.g., glycine.

The effects of these amino acid substitutions or deletions or additions may be assessed for derivatives of the Nrg-1 protein by analyzing the ability of the derivative proteins to complement the sensitivity to DNA cross-linking agents exhibited by Nrg-1 cells. These assays may be performed by transfecting DNA molecules encoding the derivative proteins into Nrg-1 cells as described above.

The Nrg-1 gene, Bos gene, Nrg-1 cDNA, Eos cDNA, DNA molecules derived therefrom and the protein encoded by the cDNA and derivative DNA molecules may be utilized in aspects of both the study of Nrg-1 (and Eos) and for diagnostic and therapeutic applications related to these proteins. Utilities of the present invention include, but are not limited to, those utilities described in the examples presented herein. Those skilled in the art will recognize that the utilities herein described are not limited to the specific experimental modes and materials presented and will appreciate the wider potential utility of this invention.

The detection of specific DNA mutations may be achieved by methods such as hybridization using specific oligonucleotides (Wallace et al. (1986). Cold Spring Harbor Symp. Quant. Biol. 51:257-261), direct DNA sequencing (Church and Gilbert (1988). Proc. Natl. Acad. Sci. USA 81:1991-1995), the use of restriction enzymes (Flavell et al. (1978). Cell 15:25), discrimination on the basis of electrophoretic mobility in gels with denaturing reagent (Myers and Maniatis (1986). Cold Spring Harbor Symp. Quant. Biol. 51:275-284), RNase protection (Myers et al. (1985). Science 230:1242), chemical cleavage (Cotton et al. (1985). Proc. Natl. Acad. Sci. USA 85:4397-4401), and the ligase-mediated detection procedure (Landegren et al., 1988).

Oligonucleotides specific to normal or mutant sequences are chemically synthesized using commercially available machines, labelled radioactively with isotopes (such as .sup.32 P) or non-radioactively (with tags such as biotin (Ward and Langer et al. Proc. Natl. Acad. Sci. USA 78:6633-6657 1981), and hybridized to individual DNA samples immobilized on membranes or other solid supports by dot-blot or transfer from gels after electrophoresis. The presence or absence of these specific sequences are visualized by methods such as autoradiography or fluorometric or colorimetric reactions (Gebeyehu et al. Nucleic Acids Res. 15:4513-4534 1987).

Sequence differences between normal and mutant forms of gene regions that correlate to functional sites as described herein, also may be revealed by the direct DNA sequencing method of Church and Gilbert (1988). Cloned DNA segments may be used as probes to detect specific DNA segments. The sensitivity of this method is greatly enhanced when combined with PCR (Stoflet et al. Science 239:491-494, 1988). In this approach, a sequencing primer which lies within the amplified sequence is used with double-stranded PCR product or single-stranded template generated by a modified PCR. The sequence determination is performed by conventional procedures with radiolabeled nucleotides or by automatic sequencing procedures with fluorescent tags.

Sequence alterations may occasionally generate fortuitous restriction enzyme recognition sites or may eliminate existing restriction sites. Changes in restriction sites are revealed by the use of appropriate enzyme digestion followed by conventional gel-blot hybridization (Southern (1975). J. Mol. Biol. 98:503). DNA fragments carrying the site (either normal or mutant) are detected by their reduction in size or increase of corresponding restriction fragment numbers. Genomic DNA samples may also be amplified by PCR prior to treatment with the appropriate restriction enzyme; fragments of different sizes are then visualized under UV light in the presence of ethidium bromide after gel electrophoresis.

Genetic testing based on DNA sequence differences may be achieved by detection of alteration in electrophoretic mobility of DNA fragments in gels with or without denaturing reagent. Small sequence deletions and insertions can be visualized by high-resolution gel electrophoresis. For example, a PCR product with small deletions is clearly distinguishable from a normal sequence on an 8% non-denaturing polyacrylamide gel (WO 91/10734, Nagamine et al. Am. J. Hum. Genet. 45:337-339 1989). DNA fragments of different sequence compositions may be distinguished on denaturing formamide gradient gels in which the mobilities of different DNA fragments are retarded in the gel at different positions according to their specific “partial-melting” temperatures (Myers et al. Science 230:1242 1985). Alternatively, a method of detecting a mutation comprising a single base substitution or other small change could be based on differential primer length in a PCR. For example, an invariant primer could be used in addition to a primer specific for a mutation. The PCR products of the normal and mutant genes can then be differentially detected in acrylamide gels.

In addition to conventional gel-electrophoresis and blot-hybridization methods, DNA fragments may also be visualized by methods where the individual DNA samples are not immobilized on membranes. The probe and target sequences may be both in solution, or the probe sequence may be immobilized (Saild et al., Proc. Nat. Acad. Sci. USA 86:6230-6234 1989). A variety of detection methods, such as autoradiography involving radioisotopes, direct detection of radioactive decay (in the presence or absence of scintillant), spectrophotometry involving calorigenic reactions and fluorometry involved fluorogenic reactions, may be used to identify specific individual genotypes.

If more than one mutation is frequently encountered in the Nrg-1 gene, a system capable of detecting such multiple mutations would be desirable. For example, a PCR with multiple, specific oligonucleotide primers and hybridization probes may be used to identify all possible mutations at the same time (Chamberlain et al. Nucl. Acids Res. 16:1141-1155 (1988)). The procedure may involve immobilized sequence-specific oligonucleotides probes (Saiki et al., Proc. Nat. Acad. Sci. USA 86:6230-6234 1989).

Every document cited is specifically incorporated in its entirety by reference.

1-34. (canceled)

35. A method for decreasing the expression of PSD-95 protein in a cell, the method comprising contacting the cell with an agent that inhibits the interaction of Nrg-1 with Eos, the inhibition of interaction resulting in a decrease in PSD-95 promoter activity in the cell.

36. The method of claim 35, wherein the agent is a nucleotide sequence encoding a polypeptide that blocks interaction between Nrg-1 and Eos.

37. The method of claim 36, wherein the nucleotide sequence encodes a polypeptide having the amino acid sequence of SEQ ID NO. 8.

38. The method of claim 35, wherein the cell is a mammalian cell.

39. The method of claim 38, wherein the cell is a neuron.

40. A method for identifying a compound that modulates the Nrg-1/Eos signaling pathway in a cell, the method comprising:
(a) combining one or more test compounds with at least one or more agents that participate in the pathway;
(b) determining the amount of PSD-95 protein expressed in the cell in the presence of the test compound; and
(c) comparing the amount of PSD-95 protein expressed in the cell in the presence of the test compound with the amount of PSD-95 protein expressed in the cell in the absence of the test compound, wherein a change in the amount of PSD-95 expression in the presence of the test compound is indicative of a compound that modulates the pathway.

41. The method of claim 40, wherein the agent that participates in the signaling pathway is a nucleotide sequence encoding Nrg-ICD or a portion thereof, the nucleotide sequence hybridizing to a nucleotide sequence having SEQ ID. No:1 in 5×SSC at 42° C.

42. The method of claim 41, wherein the test compound modulates the pathway by binding to Nrg-ICD.

43. The method of claim 41, wherein the test compound modulates the pathway by inhibiting the binding of Nrg-ICD to a binding site on Eos.

44. The method of claim of claim 41, wherein the test compound modulates translocation of Nrg-ICD into the nucleus of the cell.

45. The method of claim 44, wherein the Nrg-ICD is produced transgenically within the cell and the Nrg-ICD further comprises a conjugate of a polypeptide that is at least 90% homologous with SEQ ID NO. 1 and a detectable label.

46. The method of claim 45, wherein the Nrg-ICD further comprises a nuclear localization sequence selected from the group consisting of SEQ ID NO. 3 and SEQ ID. NO. 4.

47. The method of claim 46, wherein the detectable label is selected from the group consisting of green fluorescent protein, a chemilumiphore, an antigenic peptide sequence and a regulatory marker.

48. The method of claim 47, wherein the cell is a neuron.

49. The method of claim 44, wherein the Nrg-ICD further comprises a nuclear localization sequence comprising SEQ ID NO. 2.

50. The method of claim 49, wherein the detectable label is a regulatory marker selected from the group consisting of a promoter and an enhancer.

51. The method of claim 50, wherein the cell is a neuron.

52. A method for identifying a compound that modulates proteolysis of Nrg-1 to form Nrg-ICD, the method comprising:
(a) incubating a cellular membrane form of Nrg-1 in the presence of the compound; and
(b) detecting the formation of a carboxylic end portion of Nrg-1 that is less than approximately 60 kilodaltons in size.

53. The method of claim 52, wherein the cellular form of Nrg-1 is an intact cell and the carboxylic end portion of Nrg-1 is approximately 35 kilodaltons in size.

54. The method of claim 52, wherein the carboxylic end portion is detected by an immunologically reactive water soluble peptide.


SEQUENCE LISTING (41 sequences)

SEQ ID NO: 1 - protein, 373 aa, Homo sapiens
KTKKQRKKLHDRLRQSLRSERNNVMNMANGPHHPNPPPDNVQLVNQYVSKNIISSERVVERETETSFSTSHYTSTTHHSMTVTQTPSHSWSNGHTESILSESHSVLVSSSVENSRHTSPTGPRGRLNGIGGPREGNSFLRHARETPDSYRDSPHSERYVSAMTTPARMSPVDFHTPTSPKSPPSEMSPPVSSLTISIPSVAVSPFMDEERPLLLVTPPRLREKYDNHLQQFNSFHNNPTHESNSLPPSPLRIVEDEEYETTQEYEPAQEPPKKLTNSRRVKRTKPNGHISSRVEVDSDTSSQSTSSESETEDERTGEDTPFLSIQNPMATSLEPAAAYRLAENRTNPANRFSTPEELQARLSSVIANQDPIAV

SEQ ID NO: 2 - DNA, 3272 bp, Homo sapiens
gcggccgcagctgccgggagatgcgagcgcagaccggattgtgatcacctttccctcttcgggctgtaagagagcgagacaagccaccgaagcgaggccactccagagccggcagcggagggacccgggacactagagcagctccgagccactccagactgagcggacgctccaggtgatcgagtccacgctgcttcctgcaggcgacaggcgacgcctcccgagcagcccggccactggctcttcccctcctgggacaaacttttctgcaagcccttggaccaaacttgtcgcgcgtcaccgtcacccaaccgggtccgcgtagagcgctcatcttcggcgagatgtctgagcgcaaagaaggcagaggcaaggggaagggcaagaagaaggaccggggatcccgcgggaagcccgggcccgccgagggcgacccgagcccagcactgcctcccagattgaaagaaatgaagagccaggagtcagctgcaggctccaagctagtgctccggtgcgaaaccagctccgagtactcctcactcagattcaaatggttcaagaatgggaacgagctgaaccgcaaaaataaaccagaaaacatcaagatacagaagaagccagggaagtcagagcttcgaattaacaaagcatccctggctgactctggagagtatatgtgcaaagtgatcagcaagttaggaaatgacagtgcctctgccaacatcaccattgttgagtcaaacgagttcatcactggcatgccagcctcgactgagacagcctatgtgtcctcagagtctcccattagaatctcagtttcaacagaaggcgcaaacacttcttcatccacatcaacatccacgactgggaccagccatctcataaagtgtgcggagaaggagaaaactttctgtgtgaatgggggcgagtgcttcacggtgaaggacctgtcaaacccgtcaagatacttgtgcaagtgcccaaatgagtttactggtgatcgttgccaaaactacgtaatggccagcttctacaaagcggaggaactctaccagaagagggtgctgacaattactggcatctgtatcgccctgctggtggtcggcatcatgtgtgtggtggcctactgcaaaaccaagaagcagcggcagaagcttcatgatcggcttcggcagagtcttcggtcagaacggagcaacctggtgaacatagcgaatgggcctcaccacccaaacccaccgccagagaacgtgcagctggtgaatcaatacgtatctaaaaacgtcatctccagtgagcatattgttgagagagaagtggagacttccttttccaccagtcattacacttccacagcccatcactccacgactgtcacccagactcctagtcacagctggagtaatgggcacacggagagcgtcatttcagaaagcaactccgtaatcatgatgtcttcggtagagaacagcaggcacagcagtcccgccgggggcccacgaggacgtcttcatggcctgggaggccctcgtgataacagcttcctcaggcatgccagagaaacccctgactcctacagagactctcctcatagcgaaaggtatgtatcagccatgaccaccccggctcgtatgtcacctgtagatttccacacgccaagctcccctaaatcgcccccttcggaaatgtctccacccgtgtccagcatgacggtgtccatgccctctgtggcagtcagcccctttgtggaagaagagaggcctctgctgcttgtgacgccaccaaggctacgggagaagaaatatgatcatcacccccagcaactcaactcctttcatcacaaccctgcacatcagagtaccagcctcccccctagcccactgaggatagtggaggatgaggagtacgagacgacccaggagtatgagtcagttcaagagcccgttaagaaagtcaccaatagccggcgggccaaaagaaccaagcccaatggccacattgccaataggttggaaatggacagcaacacaagttctgtgagcagtaactcagaaagtgagacagaagacgaaagagtaggtgaagacacaccattcctgggcatacagaaccccctggcagccagccttgaggtggcccctgccttccgtctggctgagagcaggactaacccagcaggccgcttctccacacaggaggaattacaggccaggctgtctagtgtaatcgctaaccaagaccctattgctgtataaaacctaaataaacacatagattcacctgtaaaactttattttatataataaagtatttcaccttaaattaaacaatttattttattttagcagttctgcaaatagaaaacaggaagaaaaaaaaacttttataaattaaatatatgtatgtaaaaatgtgttatgtgccatatgtagcaatttttttacagtatttcaaaaacgagaaagatatcaatggtgcctttatgttctgttatgtcgagagcaagttttataaagttatggtgatttctttttcacagtatttcagcaaaacctcccatatattcagtttctgctggctttttgtgcattgcattatgatgttgactggatgtatggtttgcaaggctagcagctcgctcgtgttctctctctctctctctctctctctctctctgtctctctctctgtctctctctctctctctctctctctctctctctctctctctctctctctctctctgtctctctctctgcttcccgtagctcccaaccagtactgtcttggactggcacatccatccaaatacctttctactttgtatgaagttttctttgctttcccaatatgaaatgagttctctctactctgtcagccaaaggtttgcttcactggactctgagataatagtagacccagcagcatgctactattacgtatagcaggaaactgcaccaagtaatgtccaataataggaagaaagtaatactgtgatttaaaaaaaaaaacaaactatattattaatcagaagacagcttgctcttggtaaaaggagctaccattgactctaattttgactttttagttattgttcttgacaaagagtaacagcttcaagtacagcctagaaaaaaaaatgggttctggcctgctatcaggataaatctatcgacgtagatagattcaactcagtttcactttctgtcttgggggaaatgatccagccactcatatgacgaccaaccaaccacaggtgcctctgctccctgt

SEQ ID NO: 3 - protein, 8 aa, Homo sapiens
KTKKQRKK

SEQ ID NO: 4 - protein, 7 aa, Homo sapiens
PRLREKK

SEQ ID NO: 5 - protein, 48 aa, Homo sapiens
KTKKQRKKLHDRLRQSLRSERNNVMNMANGPHHPNPPPDNVQLVNQYV

SEQ ID NO: 6 - protein, 219 aa, Homo sapiens
SERYVSAMTTPARMSPVDFHTPTSPKSPPSEMSPPVSSLTISIPSVAVSPFMDEERPLLLVTPPRLREKYDNHLQQFNSFHNNPTHESNSLPPSPLRIVEDEEYETTQEYEPAQEPPKKLTNSRRVKRTKPNGHISSRVEVDSDTSSQSTSSESETEDERTGEDTPFLSIQNPMATSLEPAAAYRLAENRTNPANRFSTPEELQARLSSVIANQDPIAV

SEQ ID NO: 7 - protein, 116 aa, Homo sapiens
LKCDVCGMVCIGPNVLMVHKRSHTGERPFHCNQCGASFTQKGNLLRHIKLHSGEKPFKCPFCNYACRRRDALTGHLRTHSVSSPTVGKPYKCNYCGRSYKQQSTLEEHKERCHNYL

SEQ ID NO: 8 - protein, 54 aa, Homo sapiens
CEHCRILFLDHVMFTIHMGCHGFRDPFECNICGYHSQDRYEFSSHIVRGEHKVG

SEQ ID NO: 9 - protein, 65 aa, Homo sapiens
YGRKKRRQRRRCEHCRILFLDHVMFTIHMGCHGFRDPFECNICGYHSQDRYEFSSHIVRGEHKVG

SEQ ID NO: 10 - protein, 70 aa, Homo sapiens
RQIKIWFQNRRMKWKKCEHCRILFLDHVMFTIHMGCHGFRDPFECNICGYHSQDRYEFSSHIVRGEHKVG

SEQ ID NO: 11 - protein, 88 aa, Homo sapiens
DAATATRGRSAASRPTERPRAPARSASRPRRPVECEHCRILFLDHVMFTIHMGCHGFRDPFECNICGYHSQDRYEFSSHIVRGEHKVG

SEQ ID NO: 12 - protein, 4 aa, Homo sapiens
QRKK

SEQ ID NO: 13 - protein, 4 aa, Homo sapiens
QRKL

SEQ ID NO: 14 - protein, 4 aa, Homo sapiens
SPHS

SEQ ID NO: 15 - protein, 4 aa, Homo sapiens
SPHE

SEQ ID NO: 16 - DNA, 28 bp, Homo sapiens
accatgtctgagggagctggcgggaggt

SEQ ID NO: 17 - DNA, 33 bp, Homo sapiens
tcatacagcgtagtctgggacgtcgtatgggta

SEQ ID NO: 18 - DNA, 33 bp, Homo sapiens
tcacctttcactatgaggagagtctctgtagga

SEQ ID NO: 19 - DNA, 26 bp, Homo sapiens
accatgaaaaccaagaaacagcggca

SEQ ID NO: 20 - DNA, 25 bp, Homo sapiens
acctttcactatgaggagagtctct

SEQ ID NO: 21 - DNA, 28 bp, Homo sapiens
caccatgtatgtatcagccatgaccacc

SEQ ID NO: 22 - DNA, 24 bp, Homo sapiens
tacagcaatagggtcttggttagc

SEQ ID NO: 23 - DNA, 33 bp, Homo sapiens
tgcggatcccagatgagtctcagcactgaagcc

SEQ ID NO: 24 - DNA, 30 bp, Homo sapiens
ctcgagctagcccaccttatgctccccccg

SEQ ID NO: 25 - DNA, 29 bp, Homo sapiens
attagatctaggtaggagggtgacttgct

SEQ ID NO: 26 - DNA, 33 bp, Homo sapiens
taaagatctagctgggaaaagagacggttagca

SEQ ID NO: 27 - DNA, 35 bp, Homo sapiens
attagatcttccgctgtgtcaagggagaacaggcg

SEQ ID NO: 28 - DNA, 34 bp, Homo sapiens
gttaagcttactaaattccagctgtgagtaaagt

SEQ ID NO: 29 - DNA, 25 bp, Homo sapiens
  mutation (12)..(13)
gtaaggacttgaaaaccagggtgag

SEQ ID NO: 30 - DNA, 30 bp, Homo sapiens
  MUTATION (15)..(16)
cgttcacttcctggaaaaggcacaggatcc

SEQ ID NO: 31 - DNA, 26 bp, Homo sapiens
  mutation (14)..(15)
ccggacgggtatgaaaacacctgatc

SEQ ID NO: 32 - DNA, 49 bp, Homo sapiens
  mutation (11)..(12)
  mutation (38)..(39)
cagaggggtgaaaatctgacttgtccagagccagctgaaaaaagagacg

SEQ ID NO: 33 - DNA, 20 bp, Homo sapiens
ctgaggctcgggggcggaag

SEQ ID NO: 34 - DNA, 20 bp, Homo sapiens
cagggaatctccctctccat

SEQ ID NO: 35 - DNA, 20 bp, Homo sapiens
caggtaatctccctctccat

SEQ ID NO: 36 - DNA, 21 bp, Mus musculus
tgtccagagccaagctgggaa

SEQ ID NO: 37 - DNA, 21 bp, Mus musculus
cactatacagagacagtccat

SEQ ID NO: 38 - DNA, 27 bp, Homo sapiens
aggacttgggaaccagggtgaggccca

SEQ ID NO: 39 - DNA, 28 bp, Homo sapiens
ccctgaacttgctaaccgtctcttttcc

SEQ ID NO: 40 - DNA, 5 bp, Homo sapiens
gggaa

SEQ ID NO: 41 - DNA, 5 bp, Homo sapiens
  MUTATION (2)..(3)
gaaaa